(19) United States
(12) Patent Application Publication
(10) Pub. No.: US 2024/0423621 A1
(43) Pub. Date: Dec. 26, 2024

(54) DEVICES, SYSTEMS, AND METHODS FOR DURAL CLOSURE

(71) Applicant: Blake W. Berman, Fontana, CA (US)

(72) Inventors: Blake W. Berman, Escondido, CA (US); David G. Matsuura, Solana Beach, CA (US); Nelson Siu, Encinitas, CA (US)

(21) Appl. No.: 18/824,733

(22) Filed: Sep. 4, 2024

Related U.S. Application Data
(63) Continuation of application No. PCT/US2023/014387, filed on Mar. 2, 2023.
(60) Provisional application No. 63/316,899, filed on Mar. 4, 2022.

(51) Int. Cl. A61B 17/08; A61B 17/00

(52) U.S. Cl. CPC A61B 17/083; A61B 17/085; A61B 2017/00004

(57) ABSTRACT

A clip for dural closure can include a base, a first leg extending distally from the base and including a first tip, the first tip including a first plurality of digits, and a second leg extending distally from the base and including a second tip, the second tip including a second plurality of digits. The clip can include a sealant positioned on an underside of the base and configured to provide a fluid seal along at least a portion of a dural opening. The clip is transformable between an open configuration and a closed configuration, wherein the first plurality of digits of the first tip of the first leg are configured to interdigitate with the second plurality of digits of the second tip of the second leg in the closed configuration.

### INCORPORATION BY REFERENCE TO ANY PRIORITY APPLICATIONS

The present application is a continuation of International Application No. PCT/US2023/014387, entitled “DEVICES, SYSTEMS, AND METHODS FOR DURAL CLOSURE,” filed Mar. 2, 2023, which claims priority benefit of U.S. Provisional Application No. 63/316,899, entitled “DEVICES, SYSTEMS, AND METHODS FOR DURAL REPAIR,” filed Mar. 4, 2022, each of which is incorporated herein by reference in its entirety.

### BACKGROUND

### Field

The present application relates generally to spinal surgery, and more particularly, to devices, systems, and methods for closing dural openings.

### Description of the Related Art

In the course of a cranial or spinal surgical procedure, an opening in the dural tissue may form, either intentionally (e.g., an intentional durotomy), as part of the procedure, or unintentionally (e.g., an unintentional dural tear), for example due to unintended contact with a surgical instrument. If left untreated, openings in the dural tissue may result in the leakage of cerebrospinal fluid. The leakage of cerebrospinal fluid is associated with a number of health complications including headache, meningitis, and nerve complications.

### SUMMARY

Aspects of the present disclosure include devices, systems, and methods for closing dural openings.

In one aspect, a forceps for dural closure is provided. The forceps includes a first blade including a proximal region, a distal region, wherein a longitudinal axis of the distal region is offset from a longitudinal axis of the proximal region, and a polymer tip including one or more surface features configured to grasp tissue. The forceps includes a second blade including a proximal region, a distal region, wherein a longitudinal axis of the distal region of the second blade is offset from a longitudinal axis of the proximal region of the second blade, and a polymer tip including one or more surface features configured to grasp tissue. The proximal region of the first blade and the proximal region of the second blade form a handle. The first blade and the second blade are dimensioned for use in a dural closure procedure.

In another aspect, a nerve hook is provided. The nerve hook includes a shaft including a lumen extending therethrough and an opening at a distal end of the lumen, and a retractable tip configured to deploy out of the opening at the distal end of the lumen and retract within the lumen, wherein the retractable tip is configured to deploy to a first deployment configuration and a second deployment configuration, the retractable tip being compact in the first deployment configuration and fanned in the second deployment configuration.

In another aspect, a clip for dural closure is provided. The clip includes a base, a first clip leg extending from the base and including a tip, the tip including a plurality of digits, a second clip leg extending from the base and including a tip, the tip including a plurality of digits, and a sealant coupled to the base. The clip is transformable between an open configuration and a closed configuration, wherein the plurality of digits of the tip of the first clip leg are configured to interdigitate with the plurality of digits of the tip of the second clip leg in the closed configuration.

In another aspect, a clip delivery device is provided. The clip delivery device includes a shaft including a proximal region, a distal region having a longitudinal axis offset from a longitudinal axis of the proximal region, a lumen extending through the shaft, and a distal opening at a distal end of the lumen. The clip delivery device includes a tip extending out of the distal opening and configured to hold one or more clips, and a handle coupled to a proximal end of the shaft, the handle including an actuator configured to actuate deployment of a clip from the tip, and a rotator configured to rotate the shaft relative to the handle.

In another aspect, a kit for dural closure is provided. The kit includes a sterile disposable forceps, a sterile disposable nerve hook, a sterile disposable clip delivery device, the disposable clip delivery device loaded with a plurality of clips, and a housing, wherein the sterile disposable forceps, the sterile disposable nerve hook, and the sterile disposable clip delivery device are sealed within the housing.

In another aspect, a clip for dural closure is provided. The clip includes a base, a first leg extending distally from the base and including a first tip, the first tip including a first plurality of digits, a second leg extending distally from the base and including a second tip, the second tip including a second plurality of digits, and a sealant positioned on an underside of the base and configured to provide a fluid seal along at least a portion of a dural opening, wherein the clip is transformable between an open configuration and a closed configuration, wherein the first plurality of digits of the first tip of the first leg are configured to interdigitate with the second plurality of digits of the second tip of the second leg in the closed configuration.

Each digit of the first plurality of digits can include a protrusion configured to be received within a depression of the second plurality of digits. The protrusion can be convex, and the depression is concave. The first tip and the second tip can be configured to clip tissue on opposing sides of the dural opening in the closed configuration to seal at least a portion of the dural opening without piercing or perforating the tissue. Each digit of the first plurality of digits can include an atraumatic distal end and each digit of the second plurality of digits including an atraumatic distal end. Each digit of the first plurality of digits can include a rounded distal end and each digit of the second plurality of digits can include a rounded distal end. The first plurality of digits can be n digits and the second plurality of digits can be n+1 digits. The sealant can include a collagen matrix. The sealant can be a plug coupled to the underside of the base. The sealant can extend along an entirety of the underside of the base between the first leg and the second leg. The sealant can include an adhesive material. The clip can be bioabsorbable. The clip can include a shape memory material. The clip can be biased to the closed configuration. The clip can include a first catch and a second catch, each of the first catch and the second catch configured to be grasped by a clip delivery device to transition the clip from the closed configuration to the open configuration. The first catch can be positioned on the first leg and the second catch is positioned on the second leg. A width of a proximal end of the clip can be no more than 1.5 times a width of a distal end of the clip. Each digit of the first plurality of digits can be angled distally. Each digit of the first plurality of digits can extend distally from a base of the digit to an apex and extends proximally from the apex to a distal end of the digit.

In another aspect, a nerve hook is provided. The nerve hook includes a shaft including a lumen extending therethrough and an opening at a distal end of the lumen, and a retractable tip configured to deploy out of the opening at the distal end of the lumen and retract within the lumen, wherein the retractable tip is configured to deploy to a compact configuration and an expanded configuration.

The tip can include a guide and one or more blades, wherein the one or more blades are movable between the compact configuration and the expanded configuration. The guide can include one or more slots, wherein each of the one or more blades is configured to deploy out of one of the one or more slots when the tip is transitioned from the compact configuration to the expanded configuration. The one or more blades can be configured to expand by movement of pressurized fluid or gas into the one or more blades. The guide can include a recess, and the one or more blades can be a single blade having a first lateral edge and a second lateral edge, each of the first lateral edge and the second lateral edge configured to be flush with the guide in the compact configuration and extend away from the guide in the expanded configuration. The tip can include a plurality of blades configured to rotate about a center point between the compact configuration and the expanded configuration. The nerve hook can include a plurality of concentric drivers, each of the plurality of concentric drivers coupled to one of the plurality of blades. The nerve hook can include a handle coupled to the shaft, the handle including an actuator configured to be actuated to transition the tip from the compact configuration to the expanded configuration. The actuator can be configured to be actuated to deploy the tip out of the opening at the distal end of the lumen and retract the tip within the lumen. The actuator can be a slider. The retractable tip can be configured to deploy along an axis offset from a longitudinal axis of the shaft. The retractable tip can be configured to deploy along an axis perpendicular to the longitudinal axis of the shaft. The tip can be configured to be inserted into a dural opening, and the tip can be configured to retain nerve roots within the dural opening in the expanded configuration. A distal edge of the tip can be configured to manipulate nerve roots and dural tissue in the compact configuration.

In another aspect, a nerve hook is provided. The nerve hook includes a shaft, and a tip rotatably coupled to the shaft, wherein the tip is configured to rotated from a withdrawn position to a procedure position, wherein, in the procedure position, the tip is configured to transition between a compact configuration and an expanded configuration.

The tip can include a guide and one or more blades, wherein the one or more blades are movable between the compact configuration and the expanded configuration. The guide can include one or more slots, wherein each of the one or more blades is configured to deploy out of one of the one or more slots when the tip is transitioned from the compact configuration to the expanded configuration. The one or more blades can be configured to expand by movement of pressurized fluid or gas into the one or more blades. The guide can include a recess, and the one or more blades can include a single blade having a first lateral edge and a second lateral edge, each of the first lateral edge and the second lateral edge configured to be flush with the guide in the compact configuration and extend away from the guide in the expanded configuration. The tip can include a plurality of blades configured to rotate about a center point between the compact configuration and the expanded configuration. The nerve hook can include a plurality of concentric drivers, each of the plurality of concentric drivers coupled to one of the plurality of blades. The nerve hook can include a handle coupled to the shaft, the handle including an actuator configured to be actuated to transition the tip from the compact configuration to the expanded configuration. The actuator can be configured to be actuated to cause the tip to rotate between the withdrawn position and the procedure position. The actuator can be a slider. In the withdrawn position, a longitudinal axis of the tip can be colinear with a longitudinal axis of a distal end of the shaft, and, in the procedure position, the longitudinal axis of the tip can be offset from the longitudinal axis of the distal end of the shaft. In the procedure position, the longitudinal axis of the tip can be perpendicular to the longitudinal axis of the distal end of the shaft. The tip can be configured to be inserted into a dural opening, and the tip can be configured to retain nerve roots within the dural opening in the expanded configuration. A distal edge of the tip can be configured to manipulate nerve roots and dural tissue in the compact configuration.

### BRIEF DESCRIPTION OF THE DRAWINGS

The features of the present disclosure will become more fully apparent from the following description taken in conjunction with the accompanying drawings. Understanding that these drawings depict only several embodiments in accordance with the disclosure and are not to be considered limiting of its scope, the disclosure will now be described with additional specificity and detail through use of the accompanying drawings.

FIG. 1A illustrates a perspective view of an embodiment of a forceps.

FIG. 1B illustrates an enlarged perspective view of a distal end the forceps of FIG. 1A.

FIG. 1C illustrates an enlarged perspective view of an alternative embodiment of the distal end of the forceps of FIG. 1B.

FIG. 2A illustrates a side view of an embodiment of a nerve hook.

FIG. 2B illustrates a side view of the nerve hook of FIG. 2A showing a deployed tip.

FIG. 2C illustrates an enlarged side view of a distal end of the nerve hook of FIG. 2A showing the deployed tip.

FIG. 2D illustrates an enlarged side view of a distal end of the nerve hook of FIG. 2A showing the deployed tip in a first deployment configuration.

FIG. 2E illustrates an enlarged side view of a distal end of the nerve hook of FIG. 2A showing the deployed tip in a second deployment configuration.

FIG. 3A illustrates a front view of an embodiment of a clip in an open configuration.

FIG. 3B illustrates a front view of the clip of FIG. 3A in a closed configuration.

FIG. 3C illustrate a perspective view of the clip of FIG. 3A in the open configuration.

FIG. 3D illustrates a perspective view of the clip of FIG. 3B in the closed configuration.

FIG. 3E illustrates an enlarged perspective view of tips of the clip of FIG. 3A in the closed configuration.

FIG. 3F illustrates an enlarged perspective view of an alternative embodiment of the tips of FIG. 3E in the closed configuration.

FIG. 4A illustrates a perspective view of an embodiment of a clip delivery device.

FIG. 4B illustrates an enlarged perspective view of a distal end of the clip delivery device of FIG. 4A.

FIG. 4C illustrates a perspective view of a shaft of the clip delivery device of FIG. 4A in a first position.

FIG. 4D illustrates a perspective view of the shaft of FIG. 4C in a second position.

FIG. 5A illustrates a perspective view of an embodiment of a nerve hook.

FIG. 5B illustrates a side view of the nerve hook of FIG. 5A.

FIG. 5C illustrates an enlarged front view of the nerve hook of FIG. 5A.

FIG. 5D illustrates an enlarged perspective view of a distal end of the nerve hook of FIG. 5A.

FIG. 5E illustrates an enlarged side view of the distal end of the nerve hook of FIG. 5A.

FIG. 5F illustrates a perspective view of the nerve hook of FIG. 5A showing a tip in an expanded configuration.

FIG. 5G illustrates an enlarged front view of the nerve hook of FIG. 5A showing the tip in the expanded configuration.

FIG. 5H illustrates an enlarged perspective view of the distal end of the nerve hook of FIG. 5A showing the tip in the expanded configuration.

FIG. 5I illustrates an enlarged side view of the distal end of the nerve hook of FIG. 5A showing the tip in the expanded configuration.

FIG. 5J illustrates a cross-sectional view of the nerve hook of FIG. 5A.

FIG. 5K illustrates an enlarged cross-sectional view of the distal end of the nerve hook of FIG. 5A.

FIG. 5L illustrates an enlarged cross-sectional view of a proximal portion of the nerve hook of FIG. 5A.

FIG. 6A illustrates an enlarged perspective view of a distal end of an embodiment of a nerve hook.

FIG. 6B illustrates an enlarged front view of the distal end of the nerve hook of FIG. 6A.

FIG. 6C illustrates an enlarged side view of the distal end of the nerve hook of FIG. 6A.

FIG. 6D illustrates an enlarged front view of the distal end of the nerve hook of FIG. 6A showing a tip in an expanded configuration.

FIG. 6E illustrates an enlarged front view of the distal end of the nerve hook of FIG. 6A showing the tip in the expanded configuration.

FIG. 6F illustrates an enlarged side view of the distal end of the nerve hook of FIG. 6A showing the tip in the expanded configuration.

FIG. 7A illustrates a side view of an embodiment of a nerve hook.

FIG. 7B illustrates a side view of a distal end of the nerve hook of FIG. 7A showing a tip in a procedure position.

FIG. 7C illustrates a perspective view of the distal end of the tip of FIG. 7A showing the tip in an expanded configuration.

FIG. 8A illustrates an enlarged perspective view of a distal end of an embodiment of a nerve hook.

FIG. 8B illustrates an enlarged perspective view of the distal end of the nerve hook of FIG. 8A showing a tip in an expanded configuration.

FIG. 9A illustrates a perspective view of an embodiment of a clip.

FIG. 9B illustrates another perspective view of the clip of FIG. 9A.

FIG. 9C illustrates a front view of the clip of FIG. 9A.

FIG. 9D illustrates a side view of the clip of FIG. 9A.

FIG. 9E illustrates a bottom view of the clip of FIG. 9A.

FIG. 9F illustrates a top view of the clip of FIG. 9A.

FIG. 10A illustrates a front view of an embodiment of a clip.

FIG. 10B illustrates a front view of an embodiment of a clip.

FIG. 11 illustrates a perspective view of an embodiment of a clip.

FIG. 12 illustrates a perspective view of an embodiment of a clip.

### DETAILED DESCRIPTION

As will be appreciated by one skilled in the art, there are numerous ways of carrying out the examples, improvements, and arrangements of devices, systems, and methods for closing dural openings in accordance with embodiments disclosed herein. Although reference will be made to the illustrative embodiments depicted in the drawings and the following description, these embodiments are not meant to be exhaustive of the various alternative designs and embodiments that are encompassed by the present disclosure. Those skilled in the art will readily appreciate that various modifications may be made, and various combinations can be made, without departing from the present disclosure.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, may be arranged, substituted, combined, and designed in a wide variety of different configurations, all of which are explicitly contemplated and make part of this disclosure.

Reference in the specification to “one embodiment,” “an embodiment”, or “in certain embodiments” means that a particular feature, structure, or characteristic described in connection with the embodiment is included in at least one embodiment. Moreover, the appearance of these or similar phrases throughout the specification does not necessarily mean that these phrases all refer to the same embodiment, nor are separate or alternative embodiments necessarily mutually exclusive. Various features are described herein which may be exhibited by certain embodiments and not by others. Similarly, various requirements are described which may be requirements for certain embodiments but may not be requirements for other embodiments.

In certain embodiments, devices, systems, and methods for closing dural openings. A dural opening may be an incidental or unintentional dural tear or an intentional durotomy. Closing a dural opening can include repairing an incidental or unintentional dural tear or closing an intentional durotomy). In certain embodiments, one or more of the devices, systems, and methods described herein may prevent and/or repair spinal fluid leaks.

In certain embodiments, one or more of the devices, systems, and methods described herein may facilitate grasping onto and holding the dural tissue in place during a dural closure. In certain embodiments, one or more of the devices, systems, and methods described herein may prevent or restrict nerve roots from herniating or retain herniated nerve roots during the closure of a dural opening. In certain embodiments, one or more of the devices, systems, and methods described herein may facilitate closing of a dural opening without piercing or perforating the dural tissue. Closing the dural opening without piercing or perforating the dural tissue may prevent intradural inflammation and scarring.

In certain embodiments, one or more of the devices described herein may be shaped, dimensioned, or otherwise configured for use in open, minimally invasive, microscopic, or endoscopic surgery. In certain embodiments, one or more of the devices described herein may be shaped, dimensioned, or otherwise configured for open, minimally invasive, microscopic, or endoscopic cranial or spinal surgical procedures in which dural closure is required. In certain embodiments, one or more of the devices described herein may be shaped, dimensioned, or otherwise configured to be visible in the microscopic field.

In certain embodiments, one or more of the devices described herein may be shaped, dimensioned, or otherwise configured for use through an incision having a length between 2 cm and 4 cm and a depth between 4 cm and 10 cm. In certain embodiments, one or more of the devices described herein may be shaped, dimensioned, or otherwise configured to be advanced to a surgical location through ha tubular retractor.

In certain embodiments, one or more of the devices described herein may be disposable. In certain embodiments, one or more of the devices described herein may be a part of a dural closure kit. In certain embodiments, the dural closure kit can be pre-packaged and/or pre-sterilized. For example, one or more of the devices described herein may be provided in a sterile condition within a sealed housing. In certain embodiments, the dural closure kit can include one or more of a forceps, a nerve hook, and a clip delivery device. In certain embodiments, each of the forceps, nerve hook, and clip delivery device can be disposable and/or configured for one time use. In certain embodiments, the dural closure kit may further include one or more clips for use with the clip delivery device. For example, in certain embodiments, the clip delivery device may be preloaded with the one or more clips. The clips may be in the form of staples.

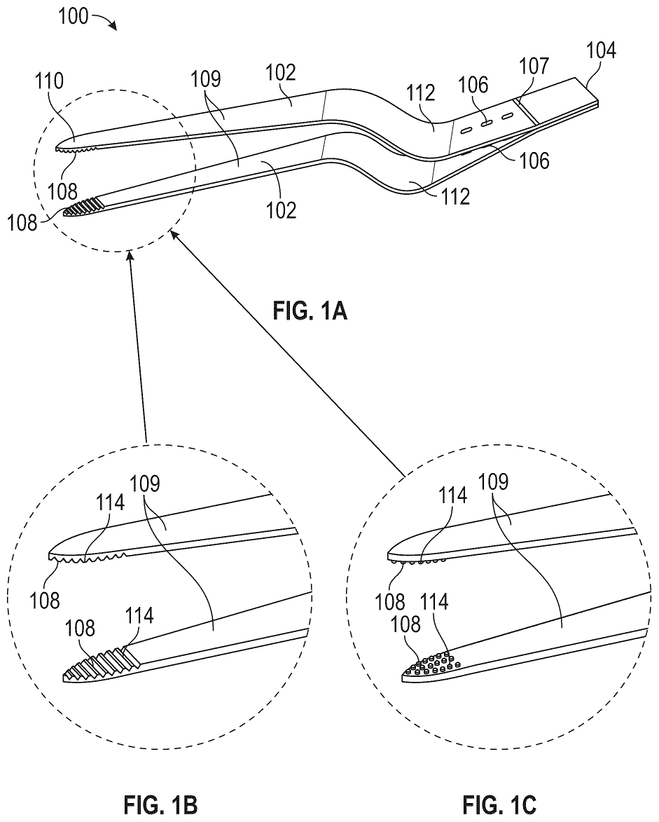

FIG. 1A illustrates an embodiment of a forceps 100. The forceps 100 can be used in a dural closure procedure. In certain embodiments, the forceps 100 can be part of a system or kit for closing a dural opening (e.g., repairing an incidental or unintentional dural tear or closing an intentional durotomy).

As shown in FIG. 1A the forceps 100 can include two blades 102. The blades 102 can be coupled to one another at a proximal end 104 of the forceps 100. Each blade 102 can include a proximal region 106. In certain embodiments, the proximal region 106 can include one or more surfaces or surface features configured to provide enhanced grasping to a user. For example, the proximal region 106 can include one or more soft polymer surfaces (e.g., rubber or polytetrafluoroethylene (PTFE)). In some embodiments, the proximal region 106 can include one or more ridges, bumps, dimples, and/or surface features or texturing to provide enhanced grasping of the proximal region by the hands of the user. The proximal regions 106 of the two blades 102 can act together as a handle 107 to allow for manipulation of the forceps 100 by a user. Each blade 102 can further include a distal region 109 having a tip 108 at a distal end 110 of the forceps 100. The tips 108 can be manipulated to move towards and away from one another, for example via manipulation of the proximal regions 106, to compress or grasp tissue and release tissue during a surgical procedure.

In certain embodiments, the forceps 100 can be bayonet forceps. The distal region 109 of each blade 102 can be offset relative to the proximal region 106. For example, a longitudinal axis of the distal region 109 can be offset from a longitudinal axis of the proximal region 106. In some embodiments, each blade 102 can include a curved or angled region 112 coupling the proximal region 106 and the distal region 109. In certain embodiments, the forceps 100 can be Yasargil-style forceps. The bayonet design can provide visibility of the dural opening during use of the forceps 100. In certain embodiments, the bayonetted design may provide improved handling/dexterity to a user in comparison to a non-bayonetted design. In alternative embodiments, forceps 100 may be curved instead of bayonetted.

In certain embodiments, the tips 108 can include surface features 114 for enhanced grasping of tissue. The surface features 114 can include bumps, ridges, teeth, dimples, and/or any other surface feature or texturing suitable for grasping tissue during a surgical procedure. For example, as shown in FIG. 1B, the surface features 114 can include a corrugated surface. In an alternative embodiment, as shown in FIG. 1C, the surface features 114 can include a plurality of protrusions.

In certain embodiments, the tips 108 can be formed of a material that enhances grasping of tissue during a surgical procedure. In certain embodiments, the tips 108 can be formed of a material that is atraumatic or provides minimal trauma to grasped dural tissue. In certain embodiments, the tips 108 can be formed at least partially of a polymer, such as rubber or PTFE, which can enhance grasping of tissue. A soft polymer, such as PTFE or rubber may also provide an atraumatic surface or provide for minimal damage to the dura in comparison to some embodiments having corrugated surfaces, for example. In certain embodiments, the tips 108 can be formed entirely of a polymer, such as rubber or PTFE. In other embodiments, the tips 108 can include a polymer layer, surface, or coating positioned to contact tissue during a surgical procedure. In certain embodiments, the forceps can be formed of plastic. In certain embodiments, the forceps 100 can be formed of plastic, and the tips 108 can be at least partially formed of a soft material, such as a soft polymer (e.g., rubber or PTFE), or can include a layer surface or coating of a soft material, such as a soft polymer (e.g., rubber or PTFE).

In certain embodiments, the forceps 100 can be dimensioned, shaped, and/or otherwise configured for use in a dural tissue closure procedure. For example, the forceps 100 can be dimensioned, shaped, and/or otherwise configured for grasping and holding dural tissue during a dural tissue closure procedure. In certain embodiments, the forceps 100 can be dimensioned, shaped, and/or otherwise configured for use under a microscope. In certain embodiments, a length between the proximal end 104 and the distal end 110 can be 20 cm, 22 cm, 23 cm, 24 cm, 25 cm, 26 cm, 27 cm, 28 cm, 30 cm, between 15 cm to 35 cm, between 20 cm to 30 cm, or any other suitable length or range. In certain embodiments, a length of each tip 108 can be 0.4 mm, 0.5 mm, 0.6 mm, 0.7 mm, 0.8 mm, 0.9 mm, 1.0 mm, between 0.1 mm and 1.2 mm, between 0.4 mm and 1.0 mm, or any other suitable length or range.

In certain embodiments, a nerve hook may be configured to handle herniated nerves. In certain embodiments, the nerve hook can be used to manipulate the nerve roots and dural tissue, for example, by contacting the nerve roots and dural tissue with a surface or edge of the nerve hook. In certain embodiments, the nerve hook can be configured to cup or otherwise receive and cradle herniated nerve roots during a dural closure procedure. In certain embodiments, the nerve hook can be inserted into a dural opening and can retain nerve roots therein to prevent the nerve roots from extending out of the dural opening.

In certain embodiments, the nerve hook may include a tip that can be used to contact the herniated nerves during a dural closure procedure. In certain embodiments, the tip may transition between different configurations, for example, to provide different functionality during different parts of a dural closure procedure. For example, in certain embodiments, the tip may have a compact configuration. In the compact configuration the tip may be used to manipulate the nerve roots and dural tissue, for example, by contacting the nerve roots and dural tissue with a surface or edge of the tip. In some embodiments, the tip may be rigid in the compact configuration.

In certain embodiments, the tip may have an expanded configuration in which at least a portion of the tip (e.g., one or more expandable tip members) expands, extends, protrudes, fans out, or otherwise transitions to provide a larger surface at the tip for contacting the nerve roots. For example, the tip may be in the form of a fan having surfaces that may fan outwardly in the expanded configuration. In some embodiments, the tip may include a guide and one or more blades, wings, protrusions, or other members that can be deployed from the guide to provide a larger surface at the tip for contacting the nerve roots. In some embodiments, in the expanded configuration, the tip can be used to capture nerve roots extending out of the dural opening and reposition the nerve roots back within the dural opening. In some embodiments, the tip can be inserted into the dural opening while in the expanded configuration and can retain nerve roots therein to prevent the nerve roots from extending out of the dural opening. In some embodiments, the tip can be inserted into the dural opening while in the compact configuration and then expanded to the expanded configuration. In some embodiments, the tip can be flexible in the expanded configuration or more flexible (e.g., less rigid) in the expanded configuration than in the compact configuration.

The tip can transition between the compact configuration and the expanded configuration in response to actuation of one or more manual actuators. For example, one or more manual actuators can be used to extend, expand, collapse, and/or retract at least a portion of the tip. In some embodiments, the one or more manual actuators may be coupled to one or more expandable tip members via one or more rods, cables, wires, etc., that may effectuate movement of the expandable tip members in response to manipulation of the one or more actuators.

In certain embodiments, the tip may additionally be configuration to transition between a withdrawn position, which may facilitate navigation of the nerve hook to the surgical location (e.g., through one or more dilators or guides) and a procedure position in which the nerve hook may be used to perform a procedure at the dural opening. In some embodiments, the tip may be retracted within a shaft of the nerve hook in the withdrawn position and advanced out of the shaft of the nerve hook in the procedure position. In some embodiments, the tip may be rotatable about a hinge or pivot point so that a longitudinal axis of the tip is colinear or generally colinear with a longitudinal axis of the shaft in the withdrawn configuration. In such embodiments, the tip may rotate about the hinge or pivot point from the withdrawn position to the procedure position. In some embodiments, in the procedure position the longitudinal axis of the tip may be offset from (e.g., transverse to, perpendicular to, or generally perpendicular to) the longitudinal axis of the shaft.

In certain embodiments, the tip of the nerve hook may include no dead space or minimal dead space to prevent catching on fragile tissue. In some embodiments, edges of the tip of the nerve hook may be shaped sized, or otherwise dimensioned not to catch on fragile tissue (e.g., rounded edges, smooth surface texture, etc.).

In some embodiments, the nerve hook may be configured to perform a minimum number of cycles of transitioning to the expanded configuration and returning to the compact configuration (e.g., 1, 2, 3, 4, 5, 6, or more cycles). In some embodiments, the nerve hook may be configured to perform a maximum number of cycles of transitioning to the expanded configuration and returning to the compact configuration (e.g., 1, 2, 3, 4, 5, 6, or more cycles). In some embodiments, the nerve hook may be configured to perform a minimum number of cycles of extending the tip from the shaft, transitioning to the expanded configuration, returning to the compact configuration, and retracting the tip within the shaft (e.g., 1, 2, 3, 4, 5, 6, or more cycles). In some embodiments, the nerve hook may be configured to perform a maximum number of cycles of extending the tip from the shaft, transitioning to the expanded configuration, returning to the compact configuration, and retracting the tip within the shaft (e.g., 1, 2, 3, 4, 5, 6, or more cycles). In some embodiments, the nerve hook may be configured to perform a minimum number of cycles of rotating the tip from the withdrawn position to the procedure position, transitioning to the expanded configuration, returning to the compact configuration, and rotating the tip from the procedure position to the withdrawn position (e.g., 1, 2, 3, 4, 5, 6, or more cycles). In some embodiments, the nerve hook may be configured to perform a maximum number of cycles of rotating the tip from the withdrawn position to the procedure position, transitioning to the expanded configuration, returning to the compact configuration, and rotating the tip from the procedure position to the withdrawn position (e.g., 1, 2, 3, 4, 5, 6, or more cycles).

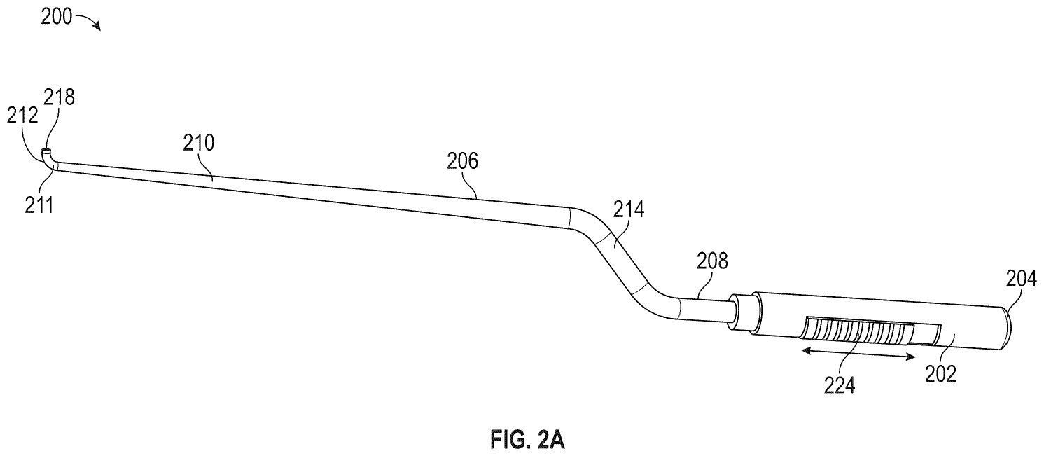

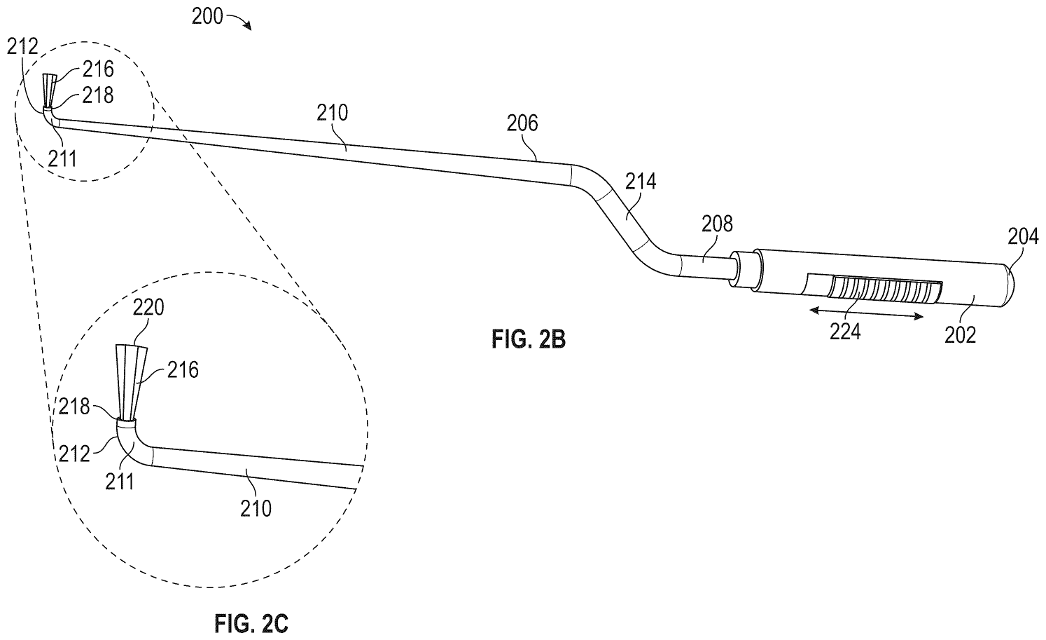

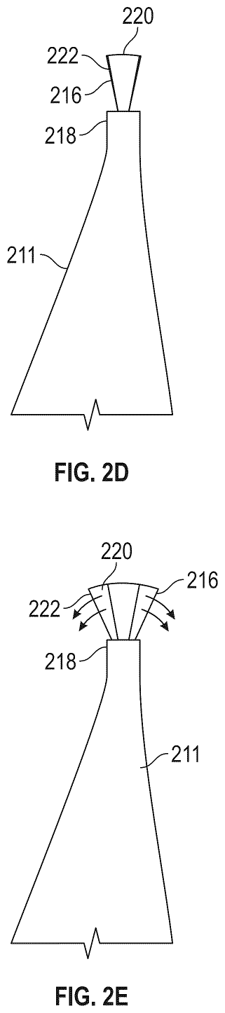

FIG. 2A illustrates an embodiment of a nerve hook 200. The nerve hook 200 can be used in a dural closure procedure. In certain embodiments, the nerve hook 200 can be part of a system or kit for closing a dural opening (e.g., repairing an incidental or unintentional dural tear or closing an intentional durotomy).

During a dural closure procedure, nerve roots can herniate out of a dural opening. In certain embodiments, the nerve hook 200 can reduce or prevent nerve roots from herniating from a dural opening. In certain embodiments, the nerve hook 200 can retain one or more herniated nerve roots.

As shown in FIG. 2A, the nerve hook 200 includes a handle 202 at a proximal end 204 of the nerve hook 200. The nerve hook 200 also includes a shaft 206. The shaft 206 can include a proximal region 208 coupled to the handle 202. The shaft 206 can also include a distal region 210 extending to and forming a distal end 212 of the nerve hook 200. The distal region 210 of the shaft 206 can include a curved distal end 211.

In certain embodiments, the shaft 206 can be bayonetted. The distal region 210 can be offset relative to the proximal region 208. For example, a longitudinal axis of the distal region 210 can be offset from a longitudinal axis of the proximal region 208. In some embodiments, the shaft 206 can include a curved or angled region 214 coupling the proximal region 208 and the distal region 210. The bayonet design can provide visibility of the dural opening during use of the nerve hook 200. In other embodiments, the shaft 206 may be curved or straight instead of bayonetted.

In certain embodiments, the shaft 206 can be in the form of a tubular shaft having an internal lumen extending therethrough. As shown in FIG. 2B, a tip 216 can be advanced out of the lumen and retracted into the lumen through an opening 218 within the distal end 211 of the shaft 206. In certain embodiments, the tip 216 can be shaped, dimensioned, or otherwise configured to manipulate nerve roots and/or dural tissue (e.g., retract dural tissue). In certain embodiments, the tip 216 can be shaped, dimensioned, or otherwise configured to reduce or retain herniated nerve roots. In certain embodiments, the tip 216 can be configured to cradle herniated nerve roots. In certain embodiments, in use, the tip 216 can be inserted into the dural opening to retain the nerve roots therein.

In certain embodiments, the tip 216 can be in the form of a fan, as shown in FIG. 2C, for example. In certain embodiments, the tip 216 can be in the form of a collapsible fan. The tip 216 can conform to a collapsed configuration when positioned within the lumen of the shaft 206. The tip 216 can deploy to one or more deployed configurations when advanced out of the shaft 206. For example, as shown in FIG. 2D, in certain embodiments, the tip 216 can deploy to a layered, unfanned, or compact configuration. In certain embodiments, when in the compact configuration, the tip 216 can allow for manipulation of the nerve roots and dural tissue, for example, by contacting the nerve roots and dural tissue with a distal edge 220 of the tip 216.

In certain embodiments, the tip 216 can deploy to an expanded or extended configuration. For example, as shown in FIG. 2E, the tip 216 can deploy to a fanned configuration. In the fanned configuration, side surfaces 222 of the tip 216 fan laterally outwards, for example circumferentially. In some embodiments, when in the fanned configuration, the tip 216 can be flat or generally flat. In some embodiments, the tip 216 can be at least partially rounded in the fanned configuration. In some embodiments, when in the fanned configuration, an inner cavity or cup is formed between the side surfaces 222. The tip 216 (for example, the inner cavity in some embodiments) can be configured to cup or otherwise receive and cradle herniated nerve roots during a dural closure procedure. The nerve roots can be retained by the tip 216 (for example, within the inner cavity) of the tip 216 during a dural closure procedure.

In certain embodiments, the tip 216 can be configured to deploy to both the compact configuration as shown in FIG. 2D and the expanded configuration (e.g., the fanned configuration) as shown in FIG. 2E. For example, in certain embodiments, the tip 216 may be in the compact configuration when partially advanced out of the opening 218 and in the expanded configuration (e.g., the fanned configuration) after additional or total advancement out of the opening 218.

In certain embodiments, the tip 216 can be configured to deploy from the opening 218 along an axis that is transverse or perpendicular to a longitudinal axis extending through the distal region 210 proximal to the curved distal end 211. In some embodiments, the curved distal end 211 can be shaped so as to cause the tip 216 to deploy along an axis that is transverse or perpendicular to the longitudinal axis extending through the distal region 210 proximal to the curved distal end 211.

In some embodiments, the tip 216 can be deployed to the compact configuration in response to a first action performed by a user (e.g., manipulation of an actuator such as a button, knob, switch, dial, lever, slider, trigger, or any other suitable actuator) and to the expanded configuration (e.g., the fanned configuration) in response to a second action performed by the user (e.g., manipulation of the same or a different actuator).

In certain embodiments, the nerve hook 200 can include an actuator 224 that can be actuated by a user to deploy the tip 216 from the opening 218 and retract the tip 216 through the opening 218. As shown in FIG. 2A, in certain embodiments, the actuator 224 can be positioned on the handle 202. In some embodiments, the actuator 224 may be a button, knob, switch, dial, lever, slider, trigger, or any other suitable actuator. As shown in FIG. 2A, the actuator 224 can be a slider that can be advanced towards the distal end 212 to cause the tip 216 to deploy. The slider can be advanced towards the proximal end 204 to cause the tip 216 to retract. In some embodiments, the actuator 224 can be actuated to cause the tip 216 to deploy in the compact configuration and further actuated to cause the tip 216 to transition to the expanded configuration (e.g., the fanned configuration). For example, in some embodiments in which the actuator 224 is a slider, the tip 216 can be retracted within the shaft 206 when the slider is in a first position. The slider can be advanced to a second position (for example, toward the distal end 212) to cause the tip 216 to deploy in the compact configuration and advanced to a third position from the second position (for example, further toward the distal end 212) to cause the tip 216 to transition to the expanded configuration (e.g., the fanned configuration). The slider can be advanced from the third position to the second position to cause the tip 216 to transition from the expanded configuration (e.g., the fanned configuration) to the compact configuration. The slider can be advanced from the second position to the first position to retract the tip 216 within the shaft 206.

In other embodiments, a first actuator may be actuated to cause the tip 216 to deploy to the compact configuration and a second actuator may be used to cause the tip 216 to transition to the expanded configuration. In such embodiments, the second actuator or another actuator (e.g., a third actuator) may be used to cause the tip 216 to transition from the expanded configuration to the compact configuration. The first actuator or another actuator (e.g., a third actuator or a fourth actuator) may be used to retract the tip 216 within the shaft 206.

In certain embodiments, the tip 216 can be formed at least partially of metal, such as nitinol. In certain embodiments, the tip 216 can be formed at least partially of a polymer, such as nylon. In certain embodiments, the tip 216 can be formed at least partially of a shape memory material, such as nitinol.

In certain embodiments, the nerve hook 200 can be dimensioned, shaped, and/or otherwise configured for use in a dural tissue closure procedure. In certain embodiments, the nerve hook 200 can be dimensioned, shaped, and/or otherwise configured for use under a microscope. In certain embodiments, a length between the proximal end 204 and the distal end 212 of the nerve hook can be 20 cm, 22 cm, 23 cm, 24 cm, 25 cm, 26 cm, 27 cm, 28 cm, 30 cm, between 15 cm to 35 cm, between 20 cm to 30 cm, or any other suitable length or range. In certain embodiments, a length of the tip 216 can be 2 mm, 3 mm, 4 mm, 5 mm, 6 mm, between 1 mm to 7 mm, between 2 mm to 8 mm, or any other suitable length or range.

In some embodiments, the nerve hook 200 can be used to capture nerve roots extending out of the dural opening and reposition the nerve roots back within the dural opening. For example, in some embodiments, the tip 216 can capture the nerve roots when in the expanded configuration (e.g., the fanned configuration). The tip 216 can then be inserted into the dural opening to reposition the nerve roots therein. In some embodiments, the tip 216 can be inserted into the dural opening while in the expanded configuration and can retain nerve roots therein to prevent the nerve roots from extending out of the dural opening. In some embodiments, the nerve hook 200 can be configured such that the tip 216 can be retained within the dural opening in the fanned configuration without additional support from a surgeon. For example, a surgeon can insert the tip 216 and release the nerve hook 200, and the nerve hook 200 can maintain its position within the dural opening. In some embodiments, when clipping or stapling of a dural opening is nearly complete, the tip 216 can be closed from the expanded configuration (e.g., the fanned configuration) to the compact configuration. Closing the tip 216 from the expanded configuration (e.g., the fanned configuration) to the compact configuration while nerve roots are cradled by the tip 216 can cause the nerve roots to be pushed back within the dural opening. After closing the tip 216 from the expanded configuration (e.g., the fanned configuration) to the compact configuration, the tip 216 can be withdrawn from the dural opening, as described herein, with minimal or no additional damage to the opening and without accidental withdrawal of additional nerve roots from the dural opening. In some embodiments, the tip 216 may be retracted prior to withdrawal from the dural opening. In contrast, traditional nerve hooks may become stuck within a dural opening near the end of a procedure to close the opening, and the attempted withdrawal of the nerve hook can lead to the withdrawal of tissue or nerve roots from the opening or the formation of a new opening.

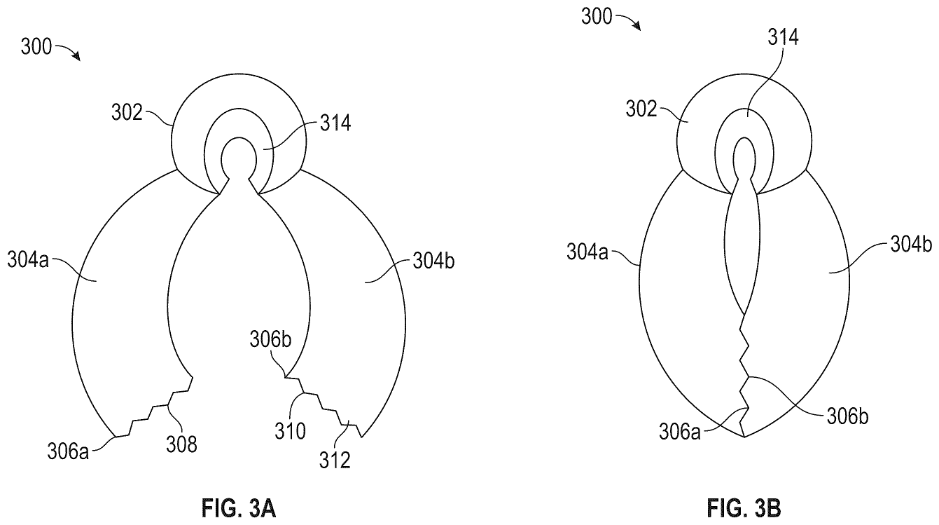

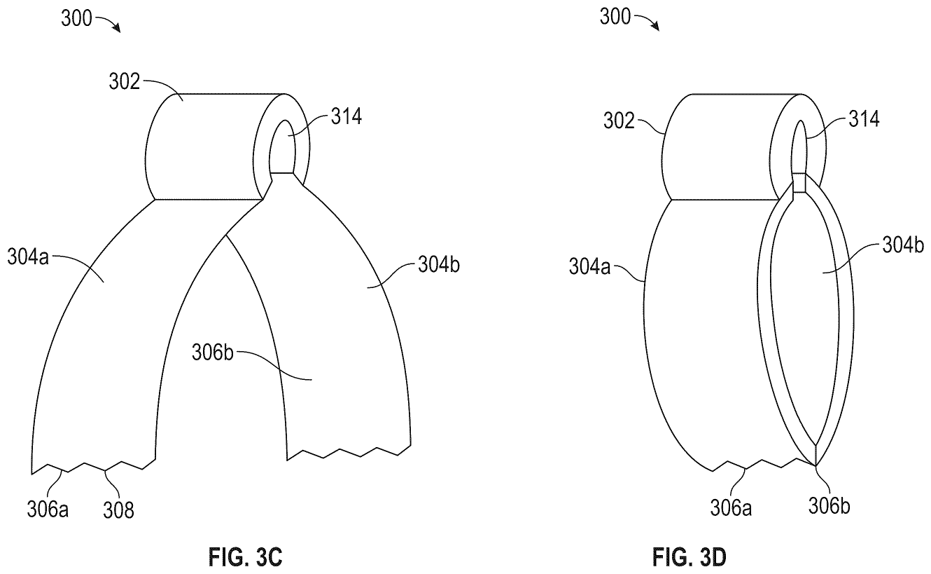

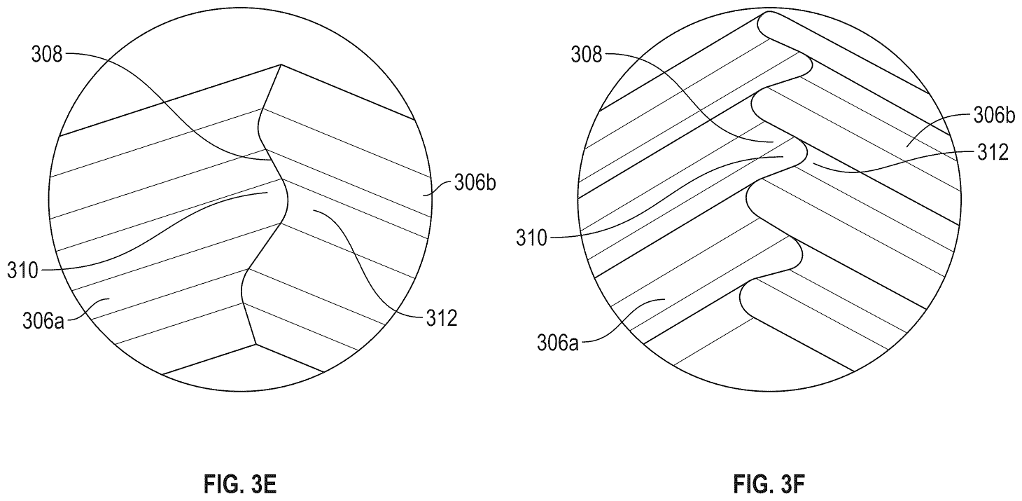

FIG. 3A illustrates an embodiment of a clip 300. The clip 300 can be used in a dural closure procedure. In certain embodiments, the clip 300 can be part of a system or kit for closing a dural opening (e.g., repairing an incidental or unintentional dural tear or closing an intentional durotomy).

In certain embodiments, the clip 300 may be in the form of a staple. As shown in FIG. 3A, the clip 300 includes a bridge or base 302. The clip further includes a pair of legs 304a and 304b extending from the base 302. The leg 304a can include a tip 306a. The leg 304b can include a tip 306b. FIG. 3A depicts the clip 300 in an open configuration. The legs 304a and 304b can be compressed towards one another to cause the tips 306a and 306b to come together in a closed configuration, as shown in FIG. 3B. FIGS. 3C and 3D illustrate side perspective views of the clip 300 in the open configuration and closed configuration, respectively.

In use, the clip 300 can be compressed towards the closed configuration about a dural opening to cause the tips 306a and 306b to pinch or clip the tissue on opposing sides of the dural opening. In certain embodiments, the tips 306a and 306b are shaped, dimensioned, or otherwise configured to interdigitate. In certain embodiments, interdigitation of the tips 306a and 306b to clip the tissue on opposing sides of the dural opening can seal the opening at the position of the clip 300. In certain embodiments, the clip 300 can be atraumatic. In certain embodiments, the tips 306a and 306b can be shaped, dimensioned, or otherwise configured to clip the tissue therebetween without piercing or perforating the tissue. For example, in certain embodiments, the tips 306a and 306b can be blunt. Closing the dural opening without piercing or perforating the dural tissue may prevent intradural inflammation (for example, arachnoiditis) and scarring.

In certain embodiments, each of the tips 306a and 306b may be in the shape of a claw. In certain embodiments, each one of the tips 306a and 306b can include a plurality of digits 308 configured to interdigitate with the digits 308 of the other one of the tips 306a and 306b. In certain embodiments, each of the digits 308 may be in the form of a protrusion 310. The tips 306a and 306b can include depressions 312 between each protrusion 310. The protrusions 310 of one of the tips 306a or 306b can be aligned with the depressions 312 of the other one of the tips 306a and 306b so that the protrusions 310 are received within the depressions 312 in the closed configuration. In certain embodiments, the protrusions 310 can be generally convex and the depressions 312 can be generally concave. In certain embodiments, each tip 306a can include 2 digits 308, 3 digits 308, 4 digits 308, 5 digits 308, or any other suitable number of digits 308. In certain embodiments, the tips 306a and 306b may each have the same number of digits 308. In other embodiments, the tips 306a and 306b may have a different number of digits 308. For example, one of the tips 306a and 306b can have 3 digits 308 and the other one of tips 306a and 306b can have 2 digits 308 or 4 digits 308.

FIG. 3E illustrates an enlarged bottom perspective view of the tips 306a and 306b interdigitating in the closed configuration. FIG. 3F illustrates an enlarged bottom perspective view of an alternative embodiment of the tips 306a and 306b, showing the tips 306a and 306b interdigitating in the closed configuration. The embodiment of FIG. 3E provides a shallower interdigitation in comparison to the embodiment of FIG. 3F. In some embodiments, a shallower interdigitation may be desirable to reduce the amount of tissue grasped in the interdigitation and prevent or reduce buckling of the tissue. In some embodiments, the tips 306a and 306b can contact one another at a plurality of contact points in the closed configuration. For example, in some embodiments, 3 points of contact may be desirable. In other embodiments, the tips 306a and 306b can contact one another at 1, 2, 4, 5, 6, or any other suitable number of contact points.

In certain embodiments, the clip 300 can include a sealant 314. As shown in FIGS. 3A-D, in some embodiments, the sealant 314 can be positioned at least partially on an underside of the base 302. In some embodiments, the sealant can be positioned across an entirety of the underside of the base 302. The sealant 314 may in the in form of a plug coupled to the underside of the base 302.

The sealant 314 can be shaped, dimensioned, positioned, or otherwise configured to form a seal over the tissue clipped by the clip 300, for example, to prevent the leakage of cerebrospinal fluid, which is under pressure in the spinal column, therefrom. In certain embodiments, the sealant 314 can be formed of a material that adheres to the dura. In certain embodiments, the sealant 314 can be formed of a material having enhanced sealing properties. In certain embodiments, the sealant 314 can be formed of a tissue that activates a healing response or biological tissue repair. For example, the sealant 314 can be formed of a tissue that activates an extradural inflammatory response and fibrotic reaction. As described above, the clips 300 can prevent intradural inflammation. In certain embodiments, the sealant 314 can be a collagen-based sealant. In certain embodiments, the sealant 314 can be in the form of a collagen matrix. In certain embodiments, the sealant 314 can provide supplemental or backup security to the closure of the dural tissue achieved by the tips 306a and 306b, for example to reduce the possibility of an incomplete or failed closure that would allow for the leakage of cerebrospinal fluid. In certain embodiments, the sealant 314 can provide a fluid-tight seal. The sealant 314 may provide a contiguous fluid seal along the closed dural opening.

In certain embodiments, the clip 300 can be dimensioned, shaped, and/or otherwise configured for use in a dural tissue closure procedure. In certain embodiments, a length of the clip 300 can be greater than a depth of the clip 300. In certain embodiments, the clips can have a length of 3 mm and a depth of 2 mm. In certain embodiments, the clips can be generally rectangular in shape. In some embodiments, the base 302 may be curved. The curvature of the base 302 may provide increased visibility of the clip during deployment.

In certain embodiments, the clip 300 can be removable. For example, the clip 300 may be removable using a clip delivery device, such as clip delivery device 400 described herein. In other embodiments, the clip 300 may be removed using an alternative clip removal tool. In some embodiments, the clip 300 may be resorbable or bioabsorbable. For example, the clip 300 may be formed of a resorbable or bioabsorbable polymer. In other embodiments, the clip 300 may be formed of a non-resorbable or non-bioabsorbable polymer. In some embodiments, the clip 300 may be removable with minimal trauma to the tissue. For example, the clip 300 may be configured to reopen or release without piercing or perforating the tissue.

In certain embodiments, the clip 300 may be pressed from the open configuration to the closed configuration, for example, using a clip delivery device, such as clip delivery device 400 as described herein. In other embodiments, the clip 300 may be biased to the closed configuration. A clip delivery device, such as clip delivery device 400 may engage the clip 300 and open the clip 300 and/or maintain the clip 300 in the open configuration. When positioned at the desired location, the clip 300 may then be released by the clip delivery device to allow the clip 300 to return to the closed configuration. In certain embodiments, the clip 300 or a portion thereof may be formed of a shape memory material, such as nitinol.

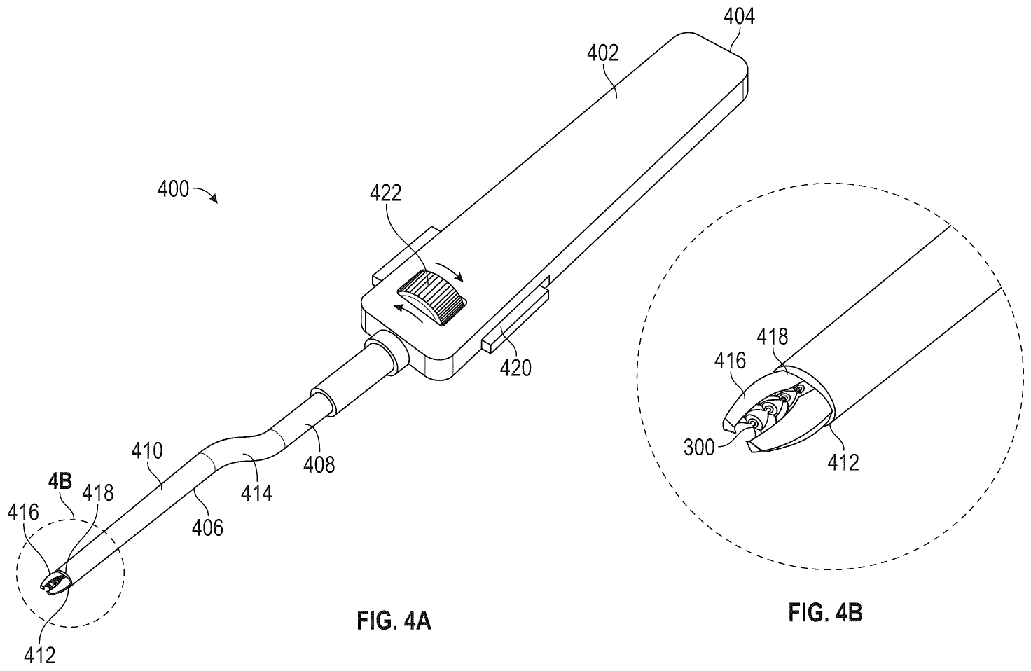

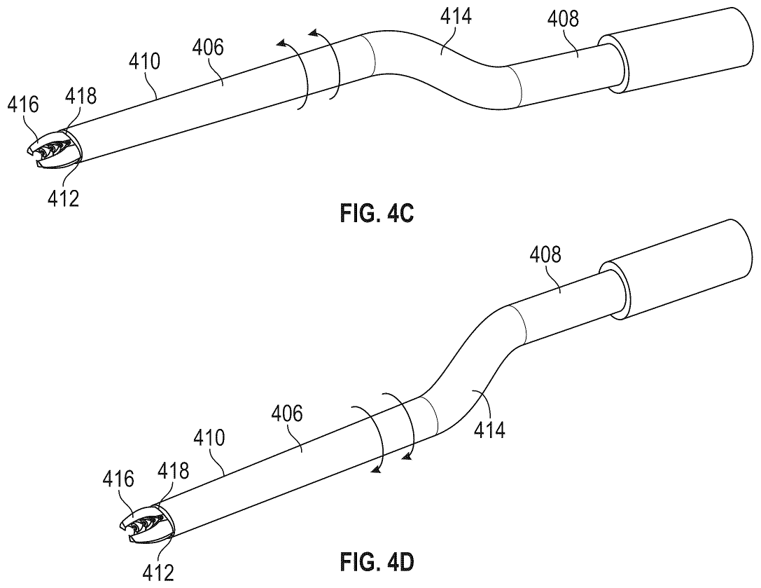

FIG. 4A illustrates an embodiment of a clip delivery device 400. The clip delivery device 400 can be used in a dural closure procedure. In certain embodiments, the clip delivery device 400 can be part of a system or kit for closing a dural opening (e.g., repairing an incidental or unintentional dural tear or closing an intentional durotomy). The clip delivery device 400 can be used to apply one or more clips 300 to a dural opening. In certain embodiments, the clip delivery device 400 may be in the form of a clip gun.

As shown in FIG. 4A, the clip delivery device 400 includes a handle 402 at a proximal end 404 of the clip delivery device 400. The clip delivery device 400 also includes a shaft 406. The shaft 406 can include a proximal region 408 coupled to the handle 402. The shaft 406 can also include a distal region 410.

In certain embodiments, the shaft 406 can be bayonetted. The distal region 410 can be offset relative to the proximal region 408. For example, a longitudinal axis of the distal region 410 can be offset from a longitudinal axis of the proximal region 408. In some embodiments, the shaft 406 can include a curved or angled region 414 coupling the proximal region 408 and the distal region 410. The bayonet design can provide visibility of the dural opening during use of the clip delivery device 400. In other embodiments, the shaft 406 can be curved instead of bayonetted.

In certain embodiments, the shaft 406 can be in the form of a tubular shaft having an internal lumen extending therethrough. In certain embodiments, a delivery tip 416 can extend from an opening 418 at a distal end 412 of the shaft 406. In certain embodiments, the tip 416 can be configured to advance outwardly to deliver a clip 300 to a desired surgical location, for example, along a dural opening. An example of advancement of a clip 300 by the tip 416 is shown in FIG. 4B. In certain embodiments, the tip 416 can be configured to close the clip 300 about the tissue at the desired surgical location. In certain embodiments, an actuator 420 may be actuated by a user to deploy the clip 300. In certain embodiments, the tip 416 can include a pincer mechanism configured to deploy the clip 300. The pincer mechanism can be activated, for example, using the actuator 420 as described in further detail below. In certain embodiments, after deployment of a clip 300, a new clip automatically enters the pincer mechanism.

In certain embodiments, the clip delivery device 400 can be configured to hold a plurality of clips 300. In some embodiments, the delivery device 400 can be configured to hold 10 clips, 15 clips, 18 clips, 20 clips, 22 clips, 25 clips, 30 clips, between 10 clips and 30 clips, between 15 clips and 25 clips, or any other suitable number of clips or range. For example, FIG. 4B shows a plurality of clips 300 positioned within the tip 416. In some embodiments, the plurality of clips 300 can be preloaded within the clip delivery device 400, for example, as part of a kit.

In certain embodiments, the clip delivery device 400 can include an actuator 420 that can be actuated by a user to deploy the clip 300 from the tip 416. As shown in FIG. 4A, in certain embodiments, the actuator 420 can be positioned on the handle 402. In certain embodiments, the actuator 420 may be a button, knob, switch, dial, lever, slider, trigger, or any other suitable actuator. As shown in FIG. 4A, the actuator 420 may be a squeezable button or trigger that may be squeezed within the grip of a user while grasping the handle 402 to actuate deployment of a clip 300. In some embodiments, the actuator 420 may include squeezable buttons or triggers on opposite sides of the handle 402. In such embodiments, both of the squeezable buttons or triggers can be squeezed simultaneously to actuate deployment of the clip 300. As described herein, in certain embodiments, the clips 300 can be deployed via a pincer mechanism, which can be actuated by the button 420.

In certain embodiments, as shown in FIGS. 4C-4D, the shaft 406 can be rotatable relative to the handle 402. In certain embodiments, the handle 402 can include an actuator 422 that can be actuated by a user to rotate or articulate the shaft 406. In certain embodiments, the actuator 422 may be a rotator, button, knob, switch, dial, lever, slider, trigger, or any other suitable actuator. As shown in FIG. 4A, in certain embodiments, the actuator 422 can be a dial. The actuator 422 can be actuated to rotate the shaft 406 relative to the handle 402 to change the position and orientation of the distal tip 416 relative to the handle 402.

In certain embodiments, the clip delivery device 400 can be dimensioned, shaped, and/or otherwise configured for use in a dural tissue closure procedure. For example, the bayonetted design may provide mechanical advantage and improved dexterity to a user in comparison to a non-bayonetted design. In certain embodiments, the clip delivery device 400 can be dimensioned, shaped, and/or otherwise configured for use under a microscope. For example, in certain embodiments, the length of the clip delivery device 400 and bayonet shape may facilitate use under a microscope. In certain embodiments, the length of the clip delivery device 400 and the thickness of the clips 300 may facilitate deployment of the clips 300 without the clips 300 falling off of the device 400 or becoming cross-clamped. In certain embodiments, the actuator 420 may be positioned between 16.5 cm and 25 cm, between 18.5 cm to 22.5 cm, or any other suitable distance from a distal most edge of the distal tip 416 or the distal most edge of the shaft 406.

In certain embodiments, the clip delivery device 400 may also function as a clip removal device to remove one or more clips, such as clip 300. For example, in certain embodiments, the actuator 420 or another actuator may be actuated to engage and retrieve the clip.

In certain embodiments, a kit for dural closure can include one or more of a forceps (e.g., forceps 100), a nerve hook (e.g., nerve hook 200, nerve hook 500, nerve hook 600, nerve hook 600a, nerve hook 600b), and a clip delivery device (e.g., clip delivery device 400). In certain embodiments, one or more clips (e.g., clips 300, clips 900, clips 900a, clips 900b, clips 900c, clips 1100) may also be included in the kit. In certain embodiments, one or more clips may be preloaded in the clip delivery device. In certain embodiments, the components within the kit can be pre-packaged and pre-sterilized. In certain embodiments, the forceps, the nerve hook, and the clip deliver device may be sterilized and provided together in a sealed container or containers for use in a dural closure procedure. In some embodiments, one or more of the components of the kit, such as the forceps, the nerve hook, and the clip delivery device can be disposable or configured for one-time use.

In certain embodiments, a method for closing a dural opening can include using a forceps (e.g., forceps 100) to grasp the torn dural tissue and hold the dural tissue in place.

In certain embodiments, the method can include using a nerve hook (e.g., nerve hook 200, nerve hook 500, nerve hook 600, nerve hook 600a, nerve hook 600b) to manipulate, reduce, and/or retain nerve roots that herniate during the method for closing a dural opening. In certain embodiments, the nerve hook may be applied at the beginning of the method. Alternatively, the nerve hook may be applied as needed, for example, when nerve roots herniate out of the dural opening.

In certain embodiments, the method can include deploying a plurality of clips (e.g., clips 300, clips 900, clips 900a, clips 900b, clips 900c, clips 1100) across the dural opening using a clip delivery device (e.g., clip delivery device 400). The clips can be deployed serially, for example, from one end of the dural opening to the other end. In certain embodiments, the forceps can be adjusted after deployment of a clip, for example, to grasp the torn tissue adjacent a location for placement of a subsequent clip. In certain embodiments, the nerve hook can be adjusted between deployment of a clip, for example to manipulate, reduce, and/or retain nerve roots adjacent a location for placement of a subsequent clip. In certain embodiments, the nerve hook can be applied to the dural opening to prevent, reduce, and/or retain herniation of nerve roots until placement of a final clip to the dural opening. In certain embodiments, prior to placement of the final clip, a tip of the nerve hook (e.g., tip 216, tip 516, tip 616, tip 616a, tip 616b) can be collapsed or otherwise transition from the expanded configuration to the compact configuration. In the compact configuration, any remaining herniated nerve roots may be manipulated and repositioned within the dura. In certain embodiments, the tip can then be retracted into the shaft of the nerve hook or rotated from a procedure position to a withdrawn position, and the nerve hook can be removed from the dural opening. In certain embodiments, the tip can be removed from the dural opening when in the compact configuration without retracting the tip within the shaft or rotating the tip to the procedure position to the withdrawn position. In certain embodiments, the tip can be rotated from the procedure position to the withdrawn position and removed from the dural opening while the tip is in the expanded configuration (e.g., without transitioning from the expanded configuration to the collapsed configuration. After the tip is removed, the final clip can be placed using the clip delivery device.

In certain embodiments, the methods described herein can be performed microscopically. In certain embodiments, the methods provided herein can be performed on openings in the spinal dura or brain dura.

In certain embodiments, the methods described herein can be performed using a pre-sterilized sealed kit including a forceps (e.g., forceps 100), a nerve hook (e.g., nerve hook 200, nerve hook 500, nerve hook 600, nerve hook 600a, nerve hook 600b), and a clip delivery device (e.g., clip delivery device 400).

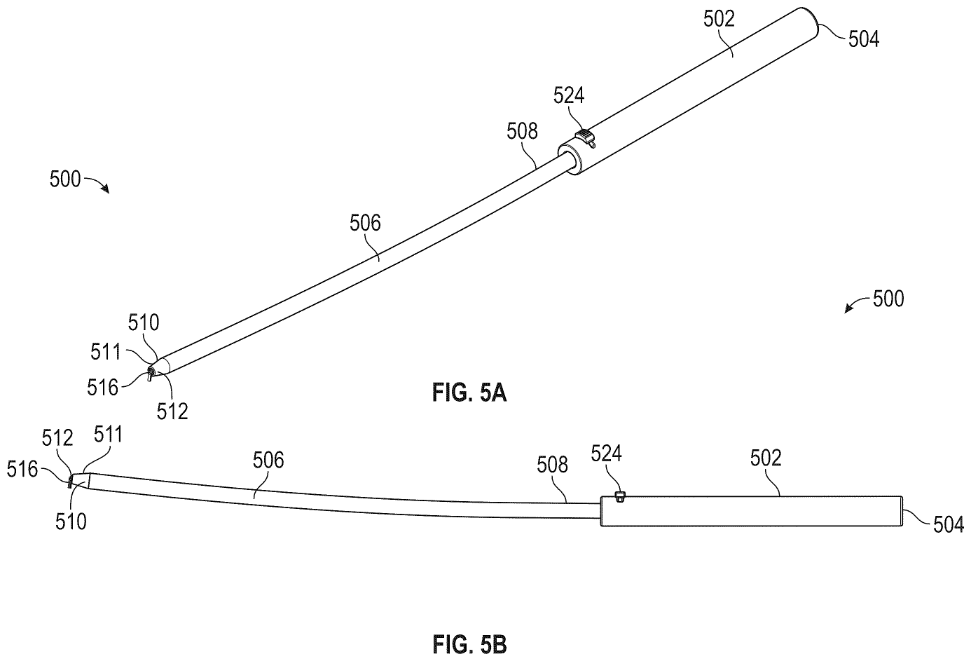

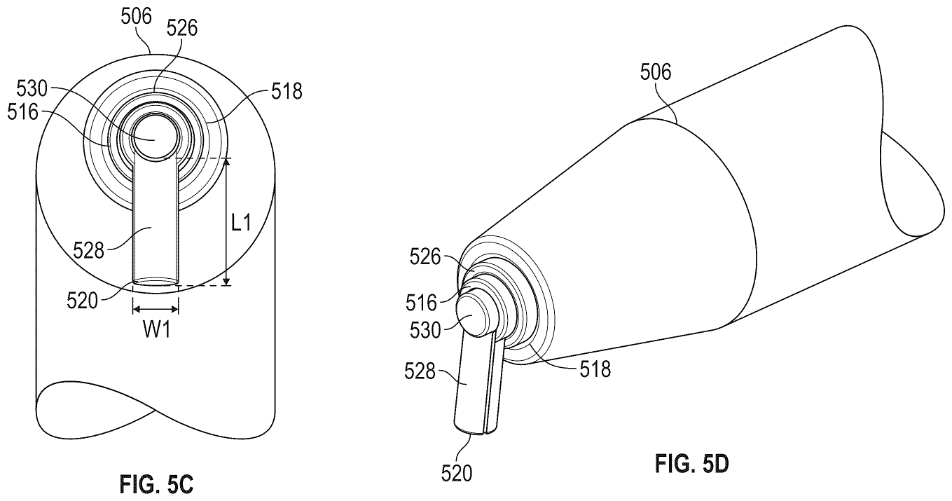

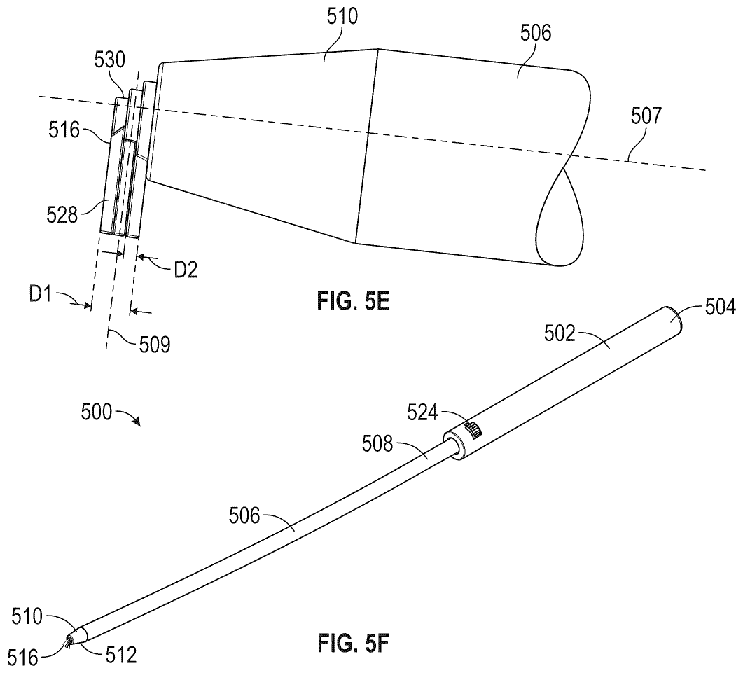

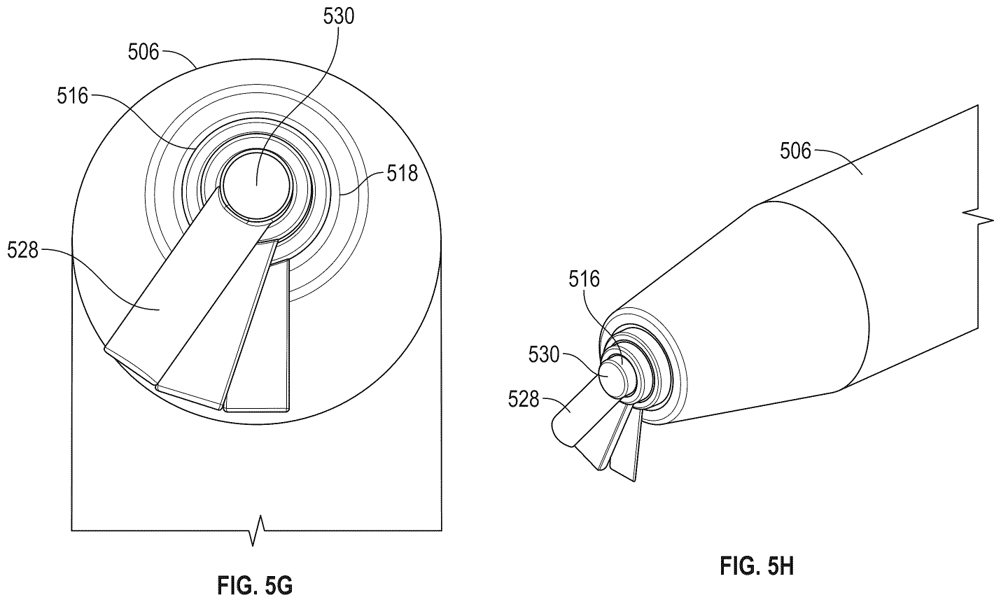

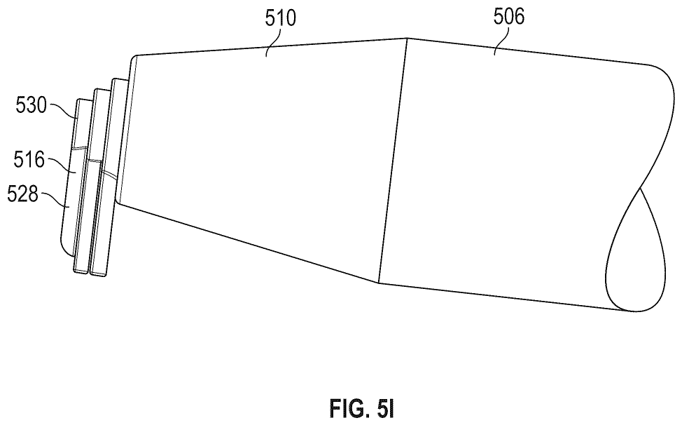

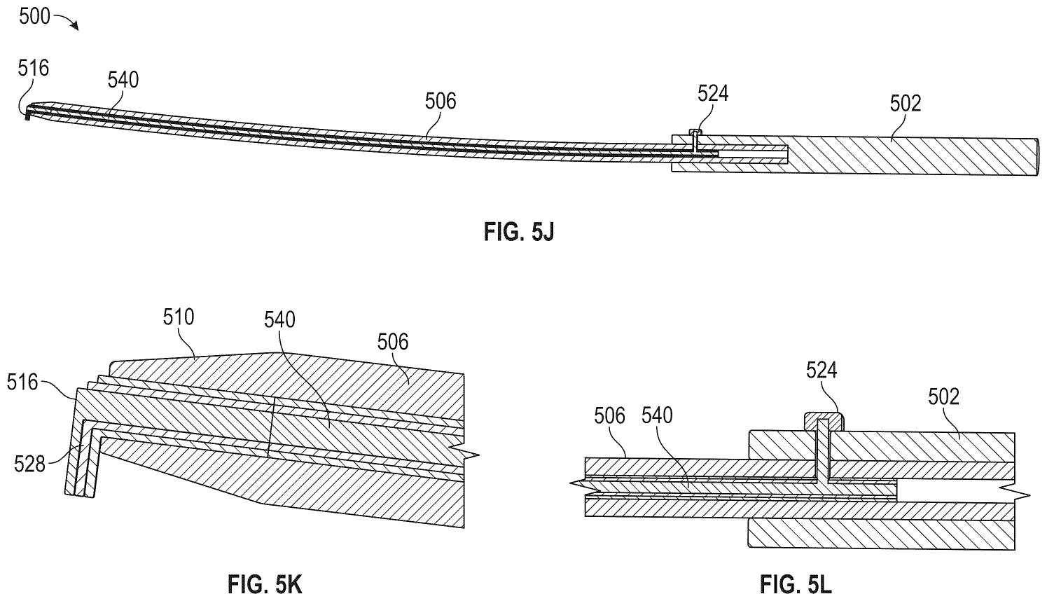

FIGS. 5A-5L illustrate an embodiment of a nerve hook 500. The nerve hook 500 can include any of the same or similar features and/or functions as the other nerve hooks described herein and vice versa.

The nerve hook 500 can be used in a dural closure procedure. In certain embodiments, the nerve hook 500 can be part of a system or kit for closing a dural opening (e.g., repairing an incidental or unintentional dural tear or closing an intentional durotomy).

As shown in FIG. 5A, the nerve hook 500 includes a handle 502 at a proximal end 504 of the nerve hook 500. The nerve hook 500 also includes a shaft 506. The shaft 506 can include a proximal region 508 coupled to the handle 502. In certain embodiments, the proximal region 508 can be generally cylindrical in shape. In certain embodiments, the shaft 506 can also include a tapered distal region 510 extending to and forming a distal end 512 of the nerve hook 500. In certain embodiments, the shaft 506 may extend axially (e.g., linearly) along a longitudinal axis from the handle 502 to the distal end 512. In certain embodiments, the shaft 506 can be in the form of a tubular shaft having an internal lumen extending therethrough.

The nerve hook 500 can include a tip 516. The tip 516 includes a proximal end 526 and a distal end 520. As shown in FIGS. 5A-5E, the tip 516 is in a compact configuration. As described in further detail herein, the tip 516 can be deployed to an expanded configuration.

As shown in FIG. 5E, a longitudinal axis 509 of the tip 516 extending between the proximal end 526 and the distal end 520 may be offset from (e.g., transverse to, perpendicular to, or generally perpendicular to) a longitudinal axis 507 of the distal region 510 of the shaft 506 (which may be the same as the longitudinal axis of the shaft). In some embodiments, the tip 516 can be rotatable (e.g., about a hinge joint) from a withdrawn position or straight position in which the longitudinal axis 509 of the tip 516 is colinear or generally colinear with the longitudinal axis 507 of the distal region 510 of the shaft and a procedure position or rotated position in which the longitudinal axis 509 of the tip 516 is offset from the longitudinal axis 507.

In certain embodiments, the tip 516 can be retracted within and advanced out of the shaft 506 in the withdrawn position. After the tip 516 is advanced out of the shaft 506, the tip 516 may be rotated to the procedure position, as shown in FIGS. 5A-5E. In other embodiments, the tip 516 may not retract within or advance out of the shaft 506. Instead the tip 516 may be positioned in the withdrawn position outside of the shaft 506 while the nerve hook is advanced to the dural opening. The tip 516 may then be rotated to the procedure position, for example, in response to actuation of an actuator (e.g., actuator 524).

In some embodiments, the tip 516 can be advanced out of the lumen and retracted into the lumen through an opening 518 within the distal end 511 of the shaft 506.

In certain embodiments, the tip 516 can be shaped, dimensioned, or otherwise configured to manipulate nerve roots and/or dural tissue (e.g., retract dural tissue). In certain embodiments, the tip 516 can be shaped, dimensioned, or otherwise configured to reduce or retain herniated nerve roots. In certain embodiments, the tip 516 can be configured to cradle herniated nerve roots. In certain embodiments, in use, the tip 516 can be inserted into the dural opening to retain the nerve roots therein.

In certain embodiments, the tip 516 can include a plurality of blades 528. The blades 528 can be configured to expand and contract around a central point 530. In certain embodiments, the blades 528 may be rigid. In certain embodiments, the blades 528 may be curved. The blades 528 may be concave or convex. In certain embodiments the blades 528 may be formed of metal. In certain embodiments, the blades 528 may nest within one another. In certain embodiments, the blades 528 may collectively form the tip 516.

In certain embodiments, the plurality of blades 528 may be positioned parallel to one another in a compact configuration as shown in FIGS. 5A-5E. In certain embodiments, when in the compact configuration, the tip 516 can allow for manipulation of the nerve roots and dural tissue, for example, by contacting the nerve roots and dural tissue with a distal edge 520 of the tip 516. In certain embodiments, the tip 516 may be maintained in the compact configuration while in the withdrawn position.

In certain embodiments, the tip 516 can deploy to an expanded, extended, or fanned configuration. For example, as shown in FIG. 5G-5I, the tip 516 can deploy to an expanded configuration in which the blades 528 expand around the central connection point 530. In certain embodiments, the blades 528 may fan out (e.g. rotate to different degrees relative to one another about the central connection point 530). When in the expanded configuration, the tip 516 (e.g., the blades 528) can be configured to receive, cradle, and/or retain herniated nerve roots during a dural closure procedure. The nerve roots can be retained by the blades 528 of the tip 516 during a dural closure procedure.

In certain embodiments, as shown in FIGS. 5J-5L, the nerve hook 500 can include a plurality of guides 540. For example, three guides 540 are illustrated in FIGS. 5J-5L. The guides can be in the form of concentric drivers or control rods that extend along the length of the shaft 506 and terminate with the blades 528. In some embodiments, the blades 528 may be “L-shaped.” In certain embodiments, the nerve hook 500 may not include a shaft 506. Instead, the guides 540 may be exposed and act as the shaft. In use, each guide 540 may move rotationally to engage its respective blade 528 at a different time, to sequentially expand the blades 528 into a fan shape in response to actuation by a user.

In certain embodiments, a first, center-most guide 540 can be integral with its respective blade 528. The center-most guide 540 and blade 528 can be one unitary piece that runs from handle 502 to the surgical field. When the blades 528 are aligned in the compact configuration, the first blade 528 can be the most distal of all the blades 528 relative to the handle 502. In such embodiments, a second guide 540 can be positioned concentrically outside of the first guide 540 and fitted with a second blade 528 at its distal end. The second blade can have a portion that is a thin-walled tube orthogonal to the longitudinal axis of the blade 528, which fits inside of the second guide 540 and outside of the first guide 540. In such embodiments, a third guide 540 can be positioned concentrically outside of the second guide 540 and fitted with a third blade 528 at its distal end. The third blade 528 can have a thin-walled tubular portion orthogonal to the long axis of the blade 528, which fits inside of the third guide 540 and outside of the second guide 540. Additional guides 540 and blades 528 may be added in this pattern to the preference of the user. In certain embodiments, each blade 528 may be coupled to a respective guide 540 at a pivot point or joint.

The nerve hook 500 can include an actuator (or actuators) 524. In some embodiments, the actuator 524 may be a button, knob, switch, dial, lever, slider, trigger, or any other suitable actuator. Similar to the actuator 224 of the nerve hook 200, the actuator (or actuators) 524 can be actuated by a user to transition the tip 516 between the compact configuration and the expanded configuration. In some embodiments, the actuator (or actuators) 524 can be actuated by a user to transition the tip 516 between the withdrawn position and the procedure position. In certain embodiments, the actuator 524 can be a rotational manual actuator. In certain embodiments, the actuator 524 can be in the form of a slider. In some embodiments in which the actuator 524 is a slider, the tip 516 can be in the withdrawn position when the slider is in a first position. The slider can be advanced to a second position (for example, advanced laterally) to cause the tip 516 to transition to the procedure position (with the tip 516 in the compact configuration) and advanced to a third position from the second position (for example, further laterally) to cause the tip 516 to transition from the compact configuration to the expanded configuration. The slider can be advanced from the third position to the second position to cause the tip 516 to transition from the expanded configuration to the compact configuration. The slider can be advanced from the second position to the first position to transition the tip 516 from the procedure position to the withdrawn position. In some embodiments, the actuator 524 or a different actuator can be used to retract the tip 516 into and advance the tip 516 out of the shaft 506.

In other embodiments, a first actuator may be actuated to cause the tip 516 to transition from the withdrawn position to the procedure position and a second actuator may be used to cause the tip 516 to transition from the compact configuration to the expanded configuration. In such embodiments, the second actuator or another actuator (e.g., a third actuator) may be used to cause the tip 516 to transition from the expanded configuration to the compact configuration. The first actuator or another actuator (e.g., a third actuator or a fourth actuator) may be used to transition the tip 516 from the procedure position to the withdrawn position.

In certain embodiments, the tip 516 can be formed at least partially of metal, such as nitinol. In certain embodiments, the tip 516 can be formed at least partially of a polymer, such as nylon. In certain embodiments, the tip 516 can be formed at least partially of a shape memory material, such as nitinol.

In certain embodiments, the nerve hook 500 can be dimensioned, shaped, and/or otherwise configured for use in a dural tissue closure procedure. In certain embodiments, the nerve hook 500 can be dimensioned, shaped, and/or otherwise configured for use under a microscope. In certain embodiments, a length between the proximal end 504 and the distal end 512 of the nerve hook can be 20 cm, 22 cm, 23 cm, 24 cm, 25 cm, 26 cm, 27 cm, 28 cm, 30 cm, between 15 cm to 35 cm, between 20 cm to 30 cm, or any other suitable length or range. In certain embodiments, a length of the tip 516 can be 2 mm, 3 mm, 4 mm, 5 mm, 6 mm, between 1 mm to 7 mm, between 2 mm to 8 mm, or any other suitable length or range.

In certain embodiments, a width W1 of each of the blades 528 can be 0.87 mm or about 0.87 mm, between 0.75 mm and 1 mm, less than 1 mm, or any other suitable width or range. A length L1 of each of the blades 528 can be 2.67 mm or about 2.67 mm, between 2.5 mm and 2.8 mm or any other suitable length or range. A depth D2 of each of the blades 528 can be 0.31 mm or about 0.31 mm, between 0.15 mm and 0.45 mm, or any other suitable depth or range. A combined depth D1 of the blades 528 can be 0.97 mm or about 0.97 mm, between 0.75 mm and 1.25 mm, less than 1 mm, or any other suitable depth or range.

In some embodiments, the nerve hook 500 can be used to capture nerve roots extending out of the dural opening and reposition the nerve roots back within the dural opening. For example, in some embodiments, the tip 516 can capture the nerve roots when in the expanded configuration. The tip 516 can then be inserted into the dural opening to reposition the nerve roots therein. In some embodiments, the tip 516 can be inserted into the dural opening while in the expanded configuration and can retain nerve roots therein to prevent the nerve roots from extending out of the dural opening. In some embodiments, the nerve hook 500 can be configured such that the tip 516 can be retained within the dural opening in the expanded configuration without additional support from a surgeon. For example, a surgeon can insert the tip 516 and release the nerve hook 500, and the nerve hook 500 can maintain its position within the dural opening. In some embodiments, when clipping or stapling of a dural opening is nearly complete, the tip 516 can be transitioned from the expanded configuration to the compact configuration. Transitioning the tip 516 from the expanded configuration to the compact configuration while nerve roots are cradled by the tip 516 can cause the nerve roots to be pushed back within the dural opening. After transitioning the tip 516 from the expanded configuration to the compact configuration, the tip 516 can be withdrawn from the dural opening, as described herein, with minimal or no additional damage to the opening and without accidental withdrawal of additional nerve roots from the dural opening. In some embodiments, the tip 516 may be retracted or rotated to the withdrawn position prior to withdrawal from the dural opening. In contrast, traditional nerve hooks may become stuck within a dural opening near the end of a procedure to close the opening, and the attempted withdrawal of the nerve hook can lead to the withdrawal of tissue or nerve roots from the opening or the formation of a new opening. In some embodiments in which the tip 516 is rotatable to a withdrawn position, the tip 516 optionally may not be transitioned to the compact configuration at the end of a dural closure procedure. Instead, the tip 516 may be rotated to the withdrawn configuration while the blades 528 are expanded, and the nerve hook 600a may be withdrawn.

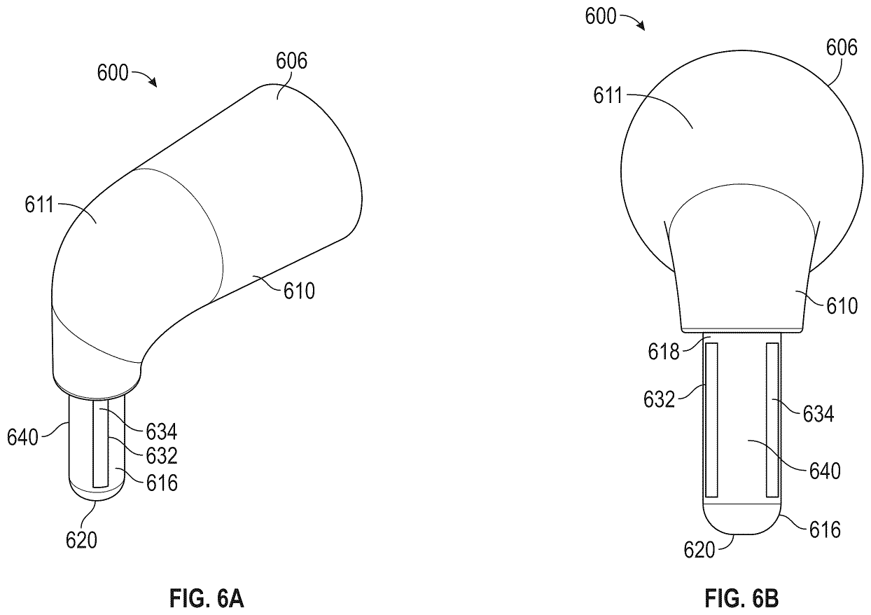

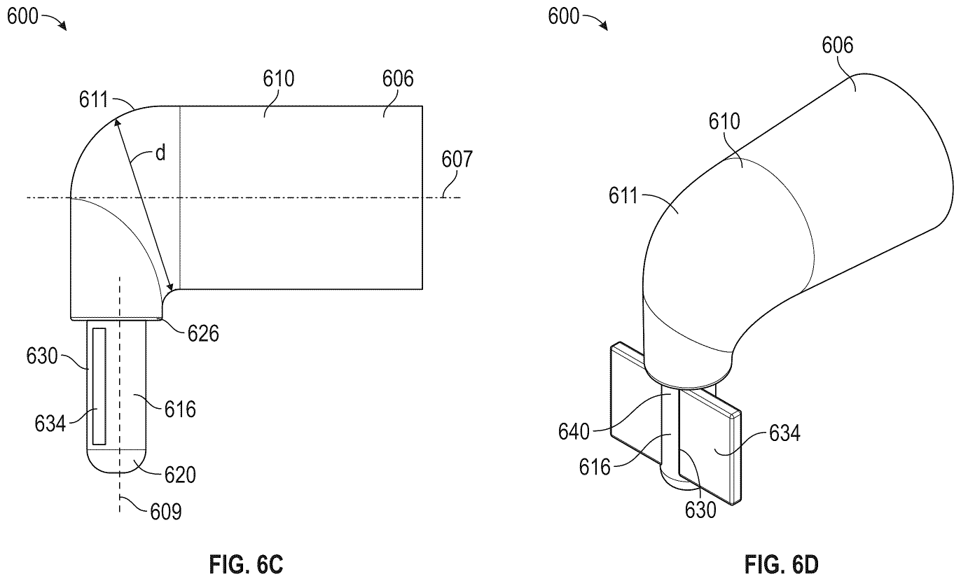

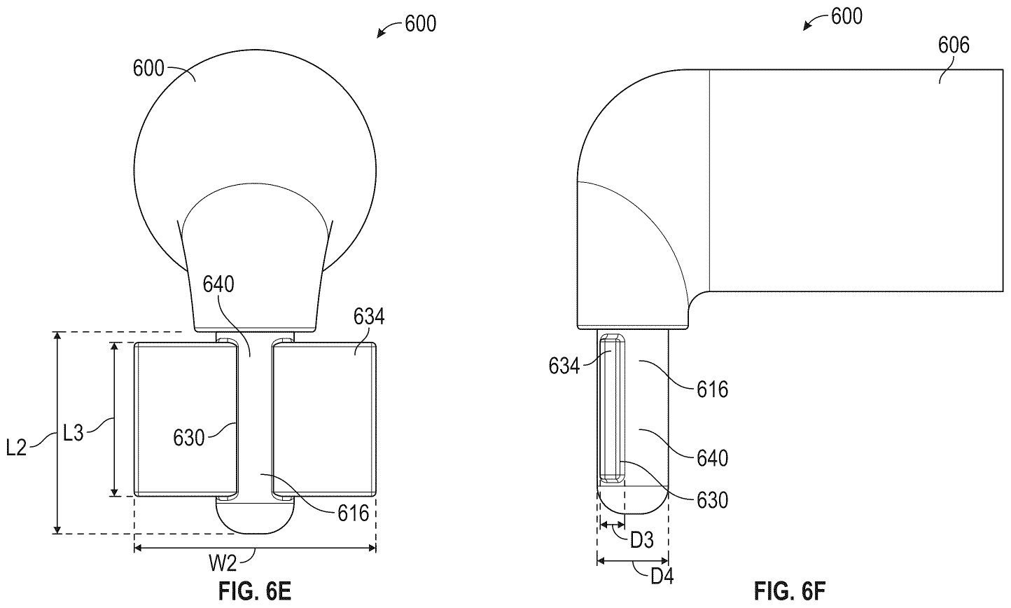

FIGS. 6A-6F illustrate an embodiment of a distal portion of a nerve hook 600. The nerve hook 600 can include any of the same or similar features and/or functions as any of the nerve hooks described herein and vice versa.

The nerve hook 600 can be used in a dural closure procedure. In certain embodiments, the nerve hook 600 can be part of a system or kit for closing a dural opening (e.g., repairing an incidental or unintentional dural tear or closing an intentional durotomy).

The nerve hook 600 also includes a shaft 606. The shaft 606 may have any of the same features and/or functions as the shaft 206 and shaft 506. The shaft 606 may also be coupled to handle, such as handle 202 and handle 502. In certain embodiments, the shaft 606 may have a diameter of no more than 3 mm, no more than 2 mm, or any other suitable diameter or range. A distal region 610 of the shaft 606 can include a curved distal end 611. In certain embodiments, the curved distal end 611 may be in the form of an elbow. In certain embodiments, a distance d between a center point of an outer arc of the curved distal end 611 and an inner arc of the curved distal end 611 can be no more than 3 mm, no more than 2 mm, or any other suitable distance or range.

In certain embodiments, the shaft 606 can be in the form of a tubular shaft having an internal lumen extending therethrough. As shown in FIGS. 6A-66F, in certain embodiments a tip 616 can be advanced out of the lumen and retracted into the lumen through an opening 618 within the distal end 611 of the shaft 606. In certain embodiments, the tip 616 can be shaped, dimensioned, or otherwise configured to manipulate nerve roots and/or dural tissue (e.g., retract dural tissue). In certain embodiments, the tip 616 can be shaped, dimensioned, or otherwise configured to reduce or retain herniated nerve roots. In certain embodiments, the tip 616 can be configured to cradle herniated nerve roots. In certain embodiments, in use, the tip 616 can be inserted into the dural opening to retain the nerve roots therein.

In certain embodiments, the tip 616 can conform to a withdrawn or retracted configuration when positioned within the lumen of the shaft 606. The tip 616 can deploy to one or more deployed configurations when advanced out of the shaft 606. For example, as shown in FIGS. 6A-6C, in certain embodiments, the tip 616 can deploy to a compact configuration. In certain embodiments, when in the compact configuration, the tip 616 can allow for manipulation of the nerve roots and dural tissue, for example, by contacting the nerve roots and dural tissue with a distal edge 620 of the tip 616. In certain embodiments, the retracted configuration of the tip 616 when retracted within the shaft 606 may be the same or generally the same as the compact configuration.

In certain embodiments, the tip 616 can deploy to an expanded or extended configuration, for example, as shown in FIGS. 6D-6F. In certain embodiments, the tip 616 can include a guide 640. In certain embodiments, the nerve hook 600 can include one or more protruding members that may extend out of the guide 640 of the tip 616 in the expanded configuration. For example, as shown in FIGS. 6D-6F, the nerve hook 600 can include one or more wings or blades 634 that extend out of the guide 640 of the tip 616 in the expanded configuration. The blades 634 may extend laterally out of the guide 640 of the tip 616. In certain embodiments, the blades 634 may be retracted or at least partially retracted within the tip 616 in the retracted configuration and/or the compact configuration. In some embodiments, the lateral edges of the blade 634 may be flush with or generally flush with the exterior of the guide 640 in the collapsed configuration and/or the compact configuration (e.g., so that that the tip 616 has a generally cylindrical exterior or otherwise rounded exterior). In certain embodiments, a cross section of the tip 616 taken perpendicular to a longitudinal axis 609 of the tip 616 can have diameter of 1 mm or less than 1 mm in the compact configuration. As shown in FIGS. 6D-6F, the nerve hook 600 can include two blades 634. In other embodiments, the nerve hook can include one, three, four, five, six, or any other suitable number of blades 634.

In certain embodiments, the guide 640 can include one or more windows or slots 632. The blades 634 may extend out of the slots 632 in the expanded configuration and retract within the slots 632 in the collapsed configuration and/or the compact configuration. In some embodiments, the guide 640 may be a polymeric hypotube. In some embodiments, the guide may be formed of metal or plastic.

In certain embodiments, the blades 634 may be inflatable. In certain embodiments, the blades 634 may be expanded and collapsed (e.g., between the compact configuration and the expanded configuration) by movement of pressurized fluid or gas into and out of the blades 634. In certain embodiments, the blades 634 may be formed of a single inflatable body that extends out of each of the slots 632. In other embodiments, the blades 634 may be formed of separate inflatable bodies. In certain embodiments, the blades 634 and guide 640 may be formed as a unitary inflatable body.

In some embodiments, the blades 634 may be formed of a metal, a shape memory alloy, a polymer, or a piezoelectric material. The blades may be expanded and retracted view wires, magnets, rods, electrical signals, and/or other means that allow for repeated expansion and retraction.

When in the expanded configuration, the tip 616 (e.g., the blades 634) can be configured to receive, cradle, and/or retain herniated nerve roots during a dural closure procedure. The nerve roots can be retained by the blades 634 of the tip 616 during a dural closure procedure.

In certain embodiments, the tip 616 can be configured to deploy from the opening 618 along an axis 609 (which may be the same as the longitudinal axis of the tip 616 in the compact configuration) that is offset from (e.g., transverse to, perpendicular to, or generally perpendicular to) a longitudinal axis 607 extending through the distal region 610 proximal to the curved distal end 611. In some embodiments, the curved distal end 611 can be shaped so as to cause the tip 616 to deploy along an axis that is offset from (e.g., transverse to, perpendicular to, or generally perpendicular to) the longitudinal axis 607 extending through the distal region 610 proximal to the curved distal end 611.

The nerve hook 600 can include an actuator (or actuators), such as for example, actuator 224 and actuator 524. In some embodiments, the actuator may be a button, knob, switch, dial, lever, slider, trigger, or any other suitable actuator. The actuator can be actuated by a user to transition the tip 616 between the retracted configuration, the compact configuration, and the expanded configuration. For example in certain embodiments, the actuator can be in the form of a slider. For example, in some embodiments in which the actuator is a slider, the tip 616 can be retracted within the shaft 606 when the slider is in a first position. The slider can be advanced to a second position to cause the tip 616 to deploy in the compact configuration and advanced to a third position from the second position to cause the tip 616 to transition to the expanded configuration. The slider can be advanced from the third position to the second position to cause the tip 616 to transition from the expanded configuration to the compact configuration. The slider can be advanced from the second position to the first position to retract the tip 616 within the shaft 606.

In other embodiments, a first actuator may be actuated to cause the tip 616 to deploy to the compact configuration and a second actuator may be used to cause the tip 616 to transition to the expanded configuration. In such embodiments, the second actuator or another actuator (e.g., a third actuator) may be used to cause the tip 616 to transition from the expanded configuration to the compact configuration. The first actuator or another actuator (e.g., a third actuator or a fourth actuator) may be used to retract the tip 616 within the shaft 606.

In certain embodiments, the tip 616 can be formed at least partially of metal, such as nitinol. In certain embodiments, the tip 616 can be formed at least partially of a polymer, such as nylon. In certain embodiments, the tip 616 can be formed at least partially of a shape memory material, such as nitinol.

In certain embodiments, the nerve hook 600 can be dimensioned, shaped, and/or otherwise configured for use in a dural tissue closure procedure. In certain embodiments, the nerve hook 600 can be dimensioned, shaped, and/or otherwise configured for use under a microscope. In certain embodiments, a length between a proximal end and a distal end of the nerve hook can be 20 cm, 22 cm, 23 cm, 24 cm, 25 cm, 26 cm, 27 cm, 28 cm, 30 cm, between 15 cm to 35 cm, between 20 cm to 30 cm, or any other suitable length or range. In certain embodiments, a length L2 of the tip 616 can be 2.5 mm or about 2.5 mm, between 2 mm and 3 mm, between 2.25 mm and 2.75 mm, or any other suitable length or range. A length L3 of a blade 634 can be 1.91 mm or about 1.91 mm, between 1.75 mm and 2.05 mm, between 1.5 mm and 2.4 mm, or any other suitable length or range. As shown in FIG. 6E, a width W2 from a lateral edge of a first blade 634 to a lateral edge of a second blade 634 may be 3 mm or about 3 mm, between 2 mm and 4 mm, between 2.5 mm and 3.5 mm, no more than 3 mm, or any other suitable width or range. A depth D3 of each blade 634 can be 0.010 mm or about 0.010 mm, between 0.005 mm and 0.015 mm, or any other suitable depth or range. A width or diameter D4 of the guide 640 (which may be the same as a width or diameter of the tip 616 when in the compact configuration) can be 0.97 mm or about 0.97 mm, between 0.75 mm and 1.25 mm, less than 1 mm, or any other suitable width or range.

In some embodiments, the nerve hook 600 can be used to capture nerve roots extending out of the dural opening and reposition the nerve roots back within the dural opening. For example, in some embodiments, the tip 616 can capture the nerve roots when in the expanded configuration. The tip 616 can then be inserted into the dural opening to reposition the nerve roots therein. In some embodiments, the tip 616 can be inserted into the dural opening while in the expanded configuration and can retain nerve roots therein to prevent the nerve roots from extending out of the dural opening. In some embodiments, the nerve hook 600 can be configured such that the tip 616 can be retained within the dural opening in the expanded configuration without additional support from a surgeon. For example, a surgeon can insert the tip 616 and release the nerve hook 600, and the nerve hook 600 can maintain its position within the dural opening. In some embodiments, when clipping or stapling of a dural opening is nearly complete, the tip 616 can be transitioned from the expanded configuration to the compact configuration. Transitioning the tip 616 from the expanded configuration to the compact configuration while nerve roots are cradled by the tip 616 can cause the nerve roots to be pushed back within the dural opening. After transitioning the tip 616 from the expanded configuration to the compact configuration, the tip 616 can be withdrawn from the dural opening, as described herein, with minimal or no additional damage to the opening and without accidental withdrawal of additional nerve roots from the dural opening. In some embodiments, the tip 616 may be retracted prior to withdrawal from the dural opening. In contrast, traditional nerve hooks may become stuck within a dural opening near the end of a procedure to close the opening, and the attempted withdrawal of the nerve hook can lead to the withdrawal of tissue or nerve roots from the opening or the formation of a new opening.

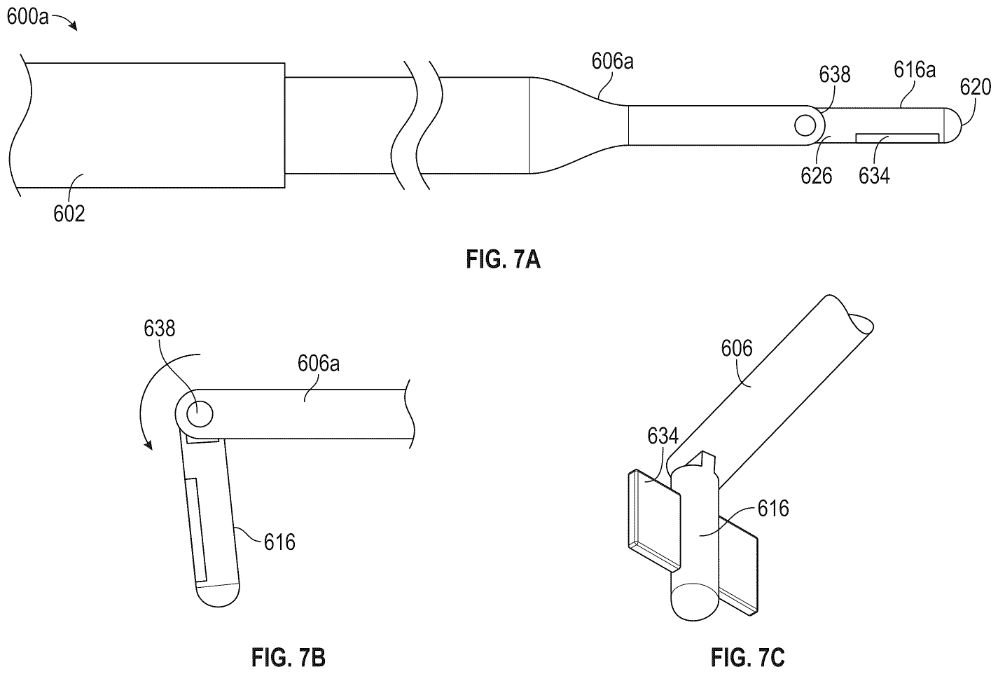

FIGS. 7A-7C illustrate a nerve hook 600a. The nerve hook 600a includes a tip 616a and a shaft 606a. The nerve hook 600a can include any of the same or similar features of functions as any of the other nerve hooks described herein. In certain embodiments, the nerve hook 600a can include generally the same features and functions as the nerve hook 600 except that the tip 616a of the nerve hook 600a does not retract within the shaft 606a. Instead, the tip 616a is configured to rotate about a pivot point or joint 638.

For example, the tip 616a can be rotatable about the joint 638 between a withdrawn position or straight position and a rotated position or procedure position. In the withdrawn position, a longitudinal axis of the tip 616a extending between a proximal end 626 and a distal end 620 can be colinear or generally colinear with the longitudinal axis of the shaft 606a. In the procedure position, the longitudinal axis of the tip 616a may be offset from (e.g., transverse to, perpendicular to, or generally perpendicular to) the longitudinal axis of the shaft 606a.

In some embodiments, the tip 616a may be positioned in the withdrawn position outside of the shaft 606a while the nerve hook 600 is advanced to the dural opening. The tip 616a may then be rotated to the procedure position as shown in FIG. 7B, for example, in response to actuation of an actuator (e.g., actuator 224 or actuator 524). The actuator may be positioned on a handle 602. After the tip 616a is rotated to the procedure position, the tip 616a may be deployed to from a compact configuration to an expanded configuration as shown in FIG. 7C, for example, in the same or a generally similar manner as described with respect to tip 616. For example, as shown in FIG. 7C, the tip 616a may include a plurality of blades 634 that extend out of and retract into a plurality of slots 630.

In some embodiments, when clipping or stapling of a dural opening is nearly complete, the tip 616a can transition from the expanded configuration to the compact configuration. Transitioning the tip 616a from the expanded configuration to the compact configuration while nerve roots are cradled by the tip 616a can cause the nerve roots to be pushed back within the dural opening. After transitioning the tip 616a from the expanded configuration to the compact configuration, the tip 616a can be withdrawn from the dural opening, as described herein, with minimal or no additional damage to the opening and without accidental withdrawal of additional nerve roots from the dural opening. In some embodiments, the tip 616a may be rotated to the withdrawn position prior to withdrawal from the dural opening. In contrast, traditional nerve hooks may become stuck within a dural opening near the end of a procedure to close the opening, and the attempted withdrawal of the nerve hook can lead to the withdrawal of tissue or nerve roots from the opening or the formation of a new opening. In some embodiments having a joint 638, the blades 634 optionally may not be transitioned to the compact configuration at the end of a dural closure procedure. Instead, the tip 616a may be rotated to the withdrawn configuration while the blades 634 are expanded, and the nerve hook 600a may be withdrawn.

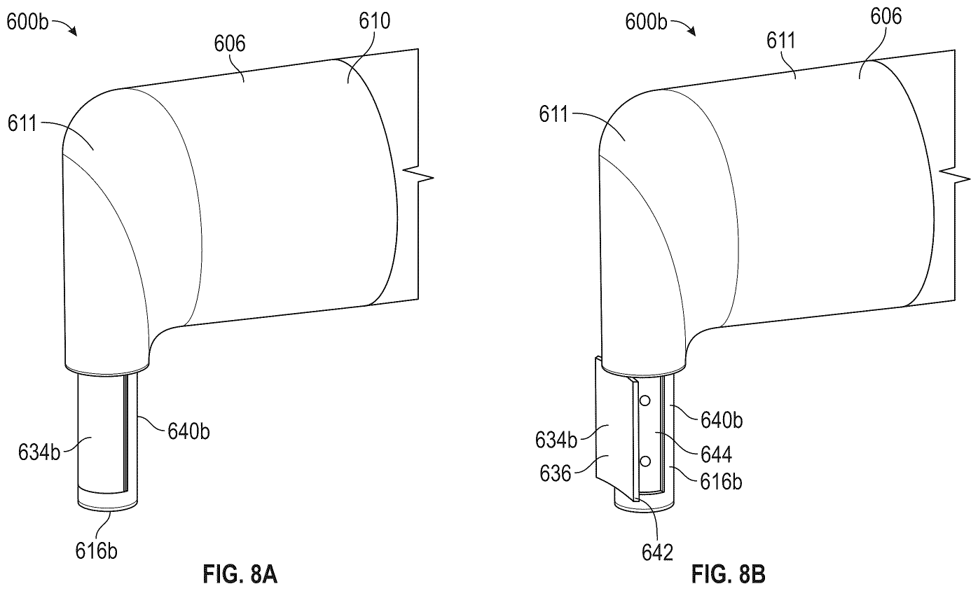

FIGS. 8A-8B illustrate distal portion of a nerve hook 600b. The nerve hook 600b includes a tip 616b and a shaft 606. The nerve hook 600b can include any of the same or similar features of functions as any of the other nerve hooks described herein. In certain embodiments, the nerve hook 600b includes generally the same features and functions as the nerve hook 600 except that the nerve hook includes a single blade 634b instead of blades 634. The blade 634b may be flush with a guide 640b of the tip 616b in the compact configuration, as shown in FIG. 8A. The guide 640b may be a polymeric hypotube. In other embodiments, the guide 640b may be a bundle of wire or polymeric steering cables, or a bundle of sliding blades that stack linearly or in a spiral pattern. The guide 640b may have one or more recesses 644 for receiving the blade 634b in the compact configuration. In some embodiments, the blade 634b may be flush with the guide 640 when retracted within the shaft 606. In other embodiments, as described herein, the tip 616b may not be retracted within the shaft 606 but may instead be coupled to the shaft 606 at a pivot point or hinge.

The blade 634b can expand to the expanded configuration as shown in FIG. 8B. In certain embodiments, portions of the blade 634b can flip, curl, extend, protrude, or project outward from the guide 604b (e.g., from the recess 644). For example, opposing lateral edges 642 of the blade 634b may extend outwardly from the guide 640b (e.g., while the blade 634b remains fixed about a central connection). In the expanded configuration, the blade 634b may be generally rectangular in shape. In certain embodiments, the blade 634b may be concave, convex, or flat. In certain embodiments, the blade 634b may be formed of a shape memory material. In certain embodiments, the blade 634b may be formed of a shape memory alloy, polymer, or piezoelectric material. The blade 634b may be a shape-set material (e.g. a rectangular shape-set material). In some embodiments, the blade 634b can include an outer surface 636 configured to contact (e.g., cup, receive, retain, cradle, etc.) the nerve roots. In the expanded configuration, the outer surface 636 may be rectangular or generally rectangular. In some embodiments, the outer surface 636 can be concave.

In certain embodiments, the blade 634b may be retained in the compact configuration by wires, magnets, electrical signals, and/or other means that allow for repeated release to the expanded configuration and retraction to the compact configuration. In some embodiments, the blade 634b may be coupled to one or more actuators (e.g., actuator 224, actuator 524, etc.) to control deployment of the blades 634b to the expanded configuration and retraction of the blade 634b to the compact configuration. In certain embodiments, the blade 634b can be coupled to the actuator(s) by one or more rods, cables, wires, magnets, etc. In certain embodiments, two wire cables may run through the center of the guide 640b and each up one side of blade 634b and back down into the guide. The two wire cables can be controlled by one or more actuators (button, knob, switch, dial, lever, slider, trigger, or any other suitable actuator) to control deployment and retraction of the blade 634b.

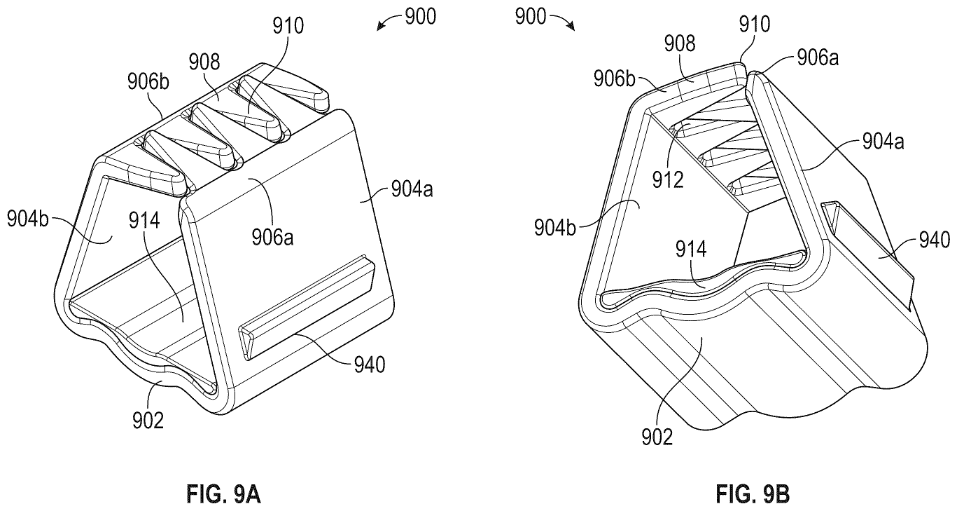

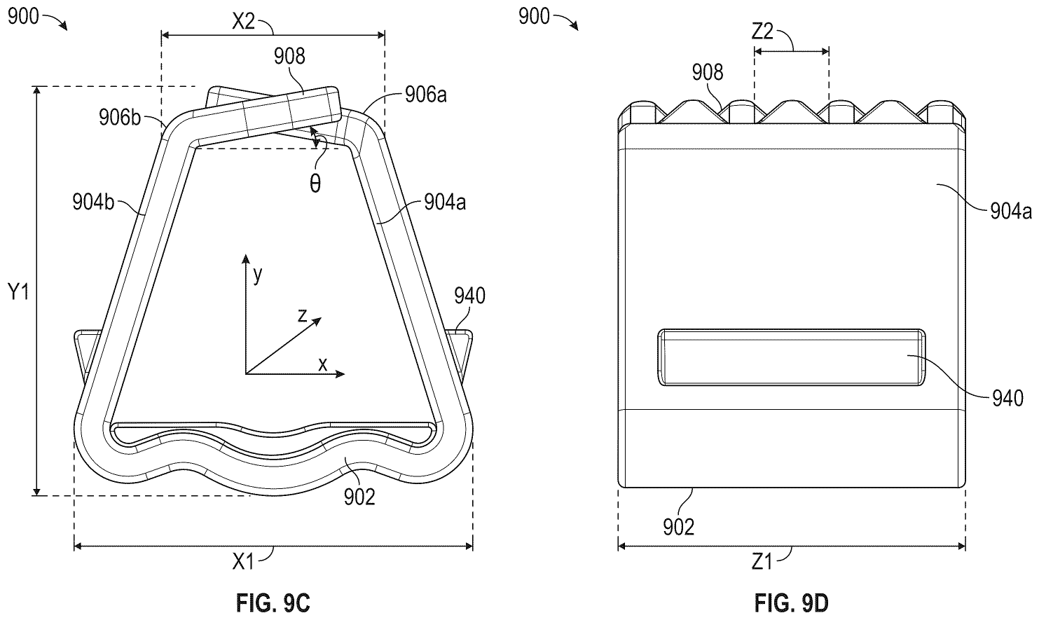

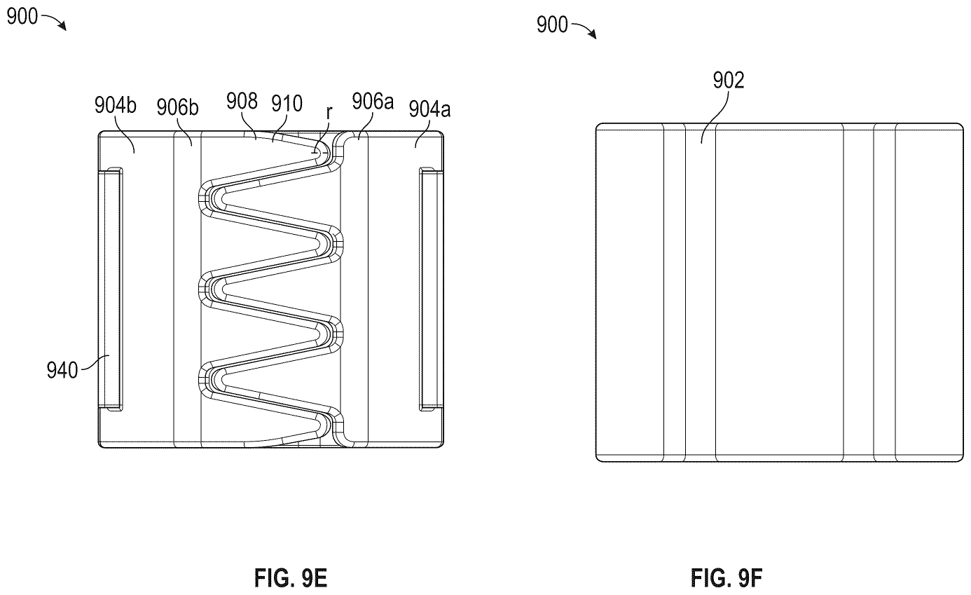

FIGS. 9A-9B illustrate an embodiment of a clip 900. The clip 900 can be used in a dural closure procedure. In certain embodiments, the clip 900 can be part of a system or kit for closing a dural opening (e.g., repairing an incidental or unintentional dural tear or closing an intentional durotomy). The clip 900 can include any of the same or similar features and/or functions as the clip 300 and vice versa.

The clip 900 can include a bridge or base 902 at a proximal end of the clip 900. The clip can further include a pair of legs 904a and 904b extending from the base 902. The leg 904a can include a tip 906a. The leg 904b can include a tip 906b. The tips 906a and 906b may be positioned at a distal end of the clip 900. FIGS. 9A-9E show the clip 900 in a closed configuration. The legs 904a and 904b can be compressed towards one another from an open configuration in which the tips 906a and 906b are separated from one another (for example, similar to the open configuration of the clip 300 shown in FIG. 3A) to cause the tips 906a and 906b to come together in the closed configuration.

In use, the clip 900 can be compressed towards the closed configuration about a dural opening to cause the tips 906a and 906b to pinch or clip the tissue on opposing sides of the dural opening. In certain embodiments, the tips 906a and 906b are shaped, dimensioned, or otherwise configured to interdigitate. In certain embodiments, interdigitation of the tips 906a and 906b to clip the tissue on opposing sides of the dural opening can seal the opening at the position of the clip 900. In certain embodiments, the tips 906a and 906b can be shaped, dimensioned, or otherwise configured to clip the tissue therebetween without piercing or perforating the tissue. For example, in certain embodiments, the tips 906a and 906b can be blunt. Closing the dural opening without piercing or perforating the dural tissue may prevent intradural inflammation (for example, arachnoiditis) and scarring.

In certain embodiments, each of the tips 906a and 906b may be in the shape of a claw. In certain embodiments, each one of the tips 906a and 906b can include a plurality of digits 908 configured to interdigitate with the digits 908 of the other one of the tips 906a and 906b. In certain embodiments, each of the digits 908 may be in the form of a tooth or protrusion. In certain embodiments, the digits may be distally angled. The tips 906a and 906b can include depressions 912 between each protrusion. The protrusions 910 of one of the tips 906a or 906b can be aligned with the depressions 912 of the other one of the tips 906a and 906b so that the protrusions 910 are received within the depressions 912 in the closed configuration. In certain embodiments, the protrusions 910 may have rounded distal ends. In certain embodiments, the protrusions 910 can be generally convex and the depressions 912 can be generally concave. In certain embodiments, each tip 906a can include 2 digits 908, 3 digits 908, 4 digits 908, 5 digits 908, or any other suitable number of digits 908. In certain embodiments, the tips 906a and 906b may each have the same number of digits 908. In other embodiments, the tips 906a and 906b may have a different number of digits 908. For example, as shown in the embodiment of FIGS. 9A-9F, the tip 906a can have 3 digits 908 and the tip 906b can have 4 digits 908. In other embodiments, one of the tips 906a and 906b can have 3 digits 908 and the other of the tips 906a and 906b can have 2 digits 908. In some embodiments, one of the tips 906a and 906b can have n digits 908 wherein n is an integer and the other of the tips 906a and 906b can have n+1 digits 908. The integer n may be 1, 2, 3, 4, 5, 6, 7, 8, or any other suitable integer.

In some embodiments, the tips 906a and 906b can contact one another at a plurality of contact points in the closed configuration. For example, in some embodiments, 3 points of contact may be desirable. In other embodiments, the tips 906a and 906b can contact one another at 1, 2, 4, 5, 6, or any other suitable number of contact points.

In certain embodiments, the clip 900 can include a sealant 914. As shown in FIGS. 9A-F, in some embodiments, the sealant 914 can be positioned at least partially on an underside of the base 902. In some embodiments, the sealant can be positioned across an entirety of the underside of the base 902. The sealant 914 may in the in form of a plug coupled to the underside of the base 902.

The sealant 914 can be shaped, dimensioned, positioned, or otherwise configured to form a seal over the tissue clipped by the clip 900, for example, to prevent the leakage of cerebrospinal fluid, which is under pressure in the spinal column, therefrom. In certain embodiments, the sealant 914 can be formed of a material that adheres to the dura (e.g., an adhesive material). In certain embodiments, the sealant 914 can be formed of a material having enhanced sealing properties. In certain embodiments, the sealant 914 can be formed of a tissue that activates a healing response or biological tissue repair. For example, the sealant 914 can be formed of a tissue that activates an extradural inflammatory response and fibrotic reaction. As described above, the clips 900 can prevent intradural inflammation. In certain embodiments, the sealant 914 can be a collagen-based sealant. In certain embodiments, the sealant 914 can be in the form of a collagen matrix. In certain embodiments, the sealant 914 can provide supplemental or backup security to the closure of the dural tissue achieved by the tips 906a and 906b, for example to reduce the possibility of an incomplete or failed closure that would allow for the leakage of cerebrospinal fluid. In certain embodiments, the sealant 914 can provide a fluid-tight seal. The sealant 314 may provide a contiguous fluid seal along the closed dural opening.

In certain embodiments, the clip 900 can be dimensioned, shaped, and/or otherwise configured for use in a dural tissue closure procedure. As shown in FIGS. 9C and 9D, the clip 900 can have a width X₁ and a width X₂ at its distal end. The widths X₁ and X₂ can be measured along an x-axis. The clip 900 can include a length or height Y₁ extending between the proximal end and the distal end. The height Y₁ can be measured along a y-axis. The clip 900 can have a depth Z₁. The depth Z₁ can be measured along a z-axis. In certain embodiments, the

In certain embodiments, a height Y₁ of the clip 900 can be greater than depth Z₁ of the clip 900. In certain embodiments, the depth Z₁ can be between 1 mm and 4 mm, between 2 mm and 3 mm, about 1 mm, about 2 mm, about 2.5 mm, about 3 mm, about 4 mm, or any other suitable depth or range. In certain embodiments, the width X₁ can be no more than 2 times wider, 1.5 times wider, or 1.25 times wider than the width X₂ or any other suitable ratio. In certain embodiments, the width X₁ can be between 1 mm and 4 mm, between 1.5 mm and 3.5 mm, or any other suitable width or range. In certain embodiments, the clips can have a height Y₁ of between 2 mm and 4 mm, about 2 mm, about 3 mm, about 4 mm, or any other suitable height or range.

As shown in FIG. 9C, in certain embodiments, the digits 908 may be angled distally by an angle θ relative to the x-axis. In certain embodiments, the angle θ can be between 1° and 20°, between 5° and 15°, about 5°, about 10°, about 15°, or any other suitable angle or range.

In certain embodiments, a wide base of each digit 908 may have a width Z₂. The width Z₂ can be between 0.35 mm and 0.55 mm, between 0.4 mm and 0.5 mm, about 0.35 mm, about 0.4 mm, about 0.45 mm, about 0.5 mm, about 0.55 mm, or any other suitable width or range.

As shown in FIG. 9E, a narrow end of each digit 908 can have a radius of between 0.05 mm and 0.15 mm, about 0.05 mm, about 0.1 mm, about 0.15 mm, or any other suitable radius or range.

In certain embodiments, the digits 908 can be sized, shaped, or otherwise configured to minimize an amount of dead space between the digits 908. In certain embodiments, a clearance around a wide base of each digit 908 may be about −0.005 mm, about −0.01 mm, about 0.015 mm, or any other suitable clearance. In certain embodiments, a clearance around a narrow end of each digit 908 may be about 0.04 mm, about 0.045 mm, about 0.05 mm, about 0.055 mm, about 0.06 mm, or any other suitable clearance.

In certain embodiments, the digits 908 may have an arced shape (e.g., the shape extends away from the x-axis and then back towards it).

In some embodiments, the base 902 may be curved. The curvature of the base 902 may provide increased visibility of the clip during deployment. The curvature of the base 902 may also provide flexion during separation of the legs 904a and 904b.

In certain embodiments, the clip 900 can be removable. For example, the clip 900 may be removable using a clip delivery device, such as clip delivery device 400 described herein. In other embodiments, the clip 900 may be removed using an alternative clip removal tool.

In certain embodiments, the clip 900 can include one or more catches 940. The catches 940 can be protrusions, openings, wings, textures surfaces, or other surface features configured to be grasped by a clip delivery device (e.g., clip delivery device 400) or a clip removal device (e.g., a forceps). As shown in FIGS. 9A-9E, in certain embodiments, the staple 900 can include a first catch 940 on the first leg 904a and a second catch 940 on the second leg 904b. As shown in FIGS. 9A-9E, in certain embodiments, the catches 940 may be triangular shaped protrusions extending laterally from the legs 904a and 904b.

In some embodiments, the clip 900 may be resorbable or bioabsorbable. For example, the clip 900 may be formed of a resorbable or bioabsorbable polymer. In other embodiments, the clip 900 may be formed of a non-resorbable or non-bioabsorbable polymer. In some embodiments, the clip 900 may be removable with minimal trauma to the tissue. For example, the clip 900 may be configured to reopen or release without piercing or perforating the tissue.

In certain embodiments, the clip 900 may be pressed from the open configuration to the closed configuration, for example, using a clip delivery device, such as clip delivery device 400 as described herein. In other embodiments, the clip 900 may be biased to the closed configuration. A clip delivery device, such as clip delivery device 400 may engage the clip 900 and open the clip 900 and/or maintain the clip 900 in the open configuration. When positioned at the desired location, the clip 900 may then be released by the clip delivery device to allow the clip 900 to return to the closed configuration (e.g., by spring action). In certain embodiments, the clip 900 or a portion thereof may be formed of a shape memory material, such as nitinol.

In certain embodiments, between ten and twenty clips 900 may be used in a dural closure procedure. In certain embodiments, the clips 900 may be pre-loaded into an elongate tubular delivery shaft of a delivery device (e.g., of delivery device 400) with a distal face of a first clip 900 against a proximal face of a second clip 900 in front of the first clip 900 (e.g., positioned to be implanted prior to the first clip 900). In certain embodiments, the delivery device may be single use. In certain embodiments, the delivery device may be configured to retrieve the clips 900 after delivery. In certain embodiments, the delivery device may be configured to be loaded with the clips 900, to deliver the clips 900, and/or to retrieve the clips 900 in a serial manner. In certain embodiments, the delivery device can include internal placement arms that can engage the catches 940 or other guide tabs on either side of each clip 900. The catches 940 or other guide tabs may be engaged, and a force may be exerted in a direction towards the base 902 to open the clip 900 (e.g., prior to application of the clip and/or retrieval).

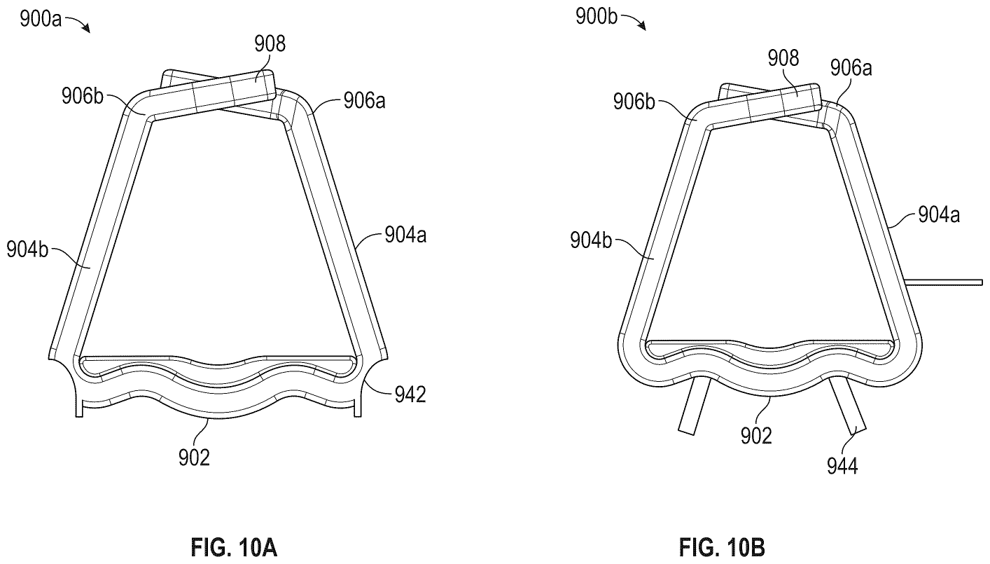

FIG. 10A illustrates an embodiment of a clip 900a. The clip 900a can include any of the same or similar features of functions as any of the other clips described herein. In certain embodiments, the clip 900a includes generally the same features and functions as the clip 900 except that the clip 900a includes catches 942 instead of catches 940. The catches 942 may be recesses, grooves, cut-outs, or other suitable surface features at the connections between the leg 904a and the base 902 and between the leg 904b and the base 902. The catches 942 can be grasped by an instrument and pressed towards one another to transition the clip 900a to the open configuration.

FIG. 10B illustrates an embodiment of a clip 900b. The clip 900b can include any of the same or similar features of functions as any of the other clips described herein. In certain embodiments, the clip 900b includes generally the same features and functions as the clip 900 except that the clip 900b includes catches 944 instead of catches 940. The catches 944 may wings, tabs, protrusions, or other suitable surface features extending proximally from the base 902. The catches 944 can be grasped by an instrument and pressed towards one another to transition the clip 900b to the open configuration.

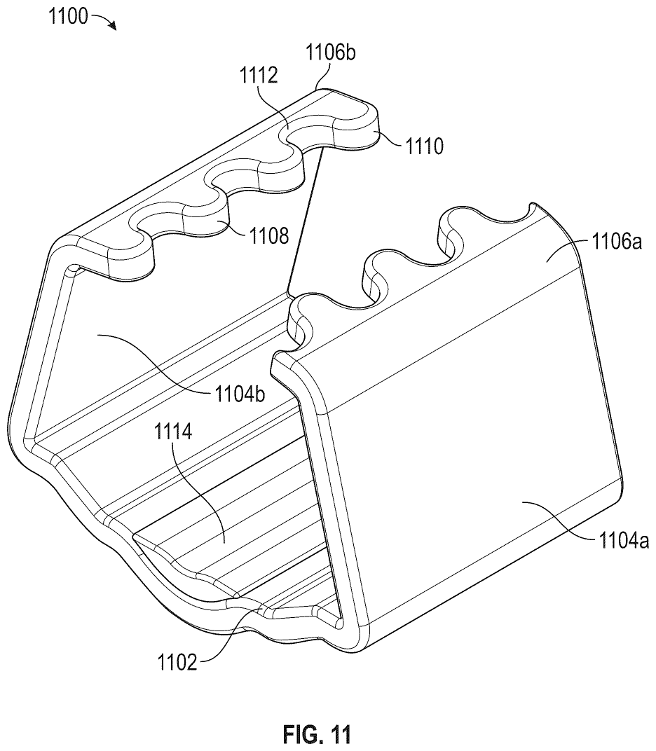

FIG. 11 illustrates an embodiment of a clip 1100. The clip 1100 can include any of the same or similar features of functions as any of the other clips described herein. The clip 1100 includes a base 1102, leg 1104a, leg 1104b, and sealant 1114. As shown in FIG. 11, clip 1100 can include tips 1106a and 1106b. Each tip 1106a and 1106b can include a plurality of digits 1108. The digits 1108 of each tip may be in the form of protrusions 1110, which may be received in corresponding depressions 1112 of the opposite tip. The protrusions 1110 and depressions 1112 may be sized, shaped, positioned, and/or otherwise configured so that there is no dead space between the digits 1108 in the closed configuration.

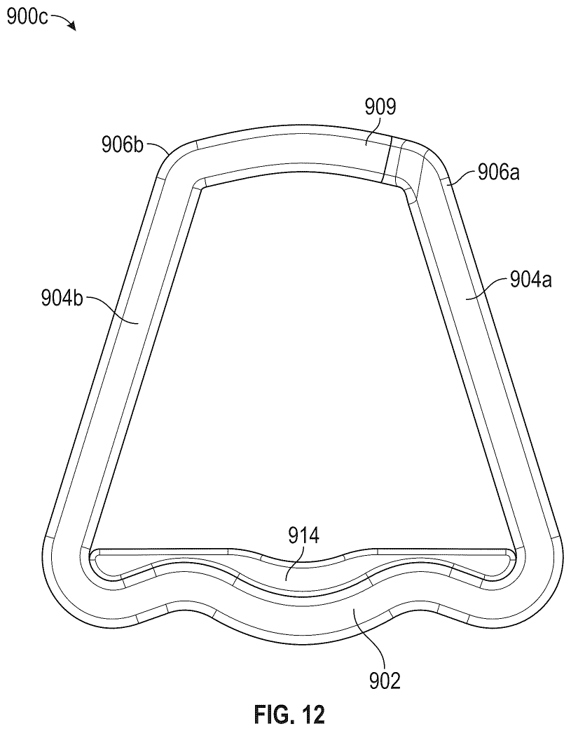

FIG. 12 illustrates an embodiment of a clip 900c. The clip 900c can include any of the same or similar features of functions as any of the other clips described herein. In certain embodiments, the clip 900c includes generally the same features and functions as the clip 900 except that the clip 900c can include digits 909 instead of digits 908. The digits 909 can include any of the same or similar features of functions except that the digits 909 can curve proximally at their distal ends. As shown in FIG. 12, each digit 909 can extend distally from its respective leg 904a and 904b to an apex and then extend proximally to the distal end of the digit 909. Each digit 909 can be generally curved or arcuate in shape having an apex between the base of the digit and its distal end. The curved or arcuate shape of the digits may provide reduce or eliminate dead space between the digits in the closed configuration.

While the systems, devices, and methods described herein are discussed primary in the context of dural closure procedures, one of skill in the art would understand that the systems, devices, and methods may be used with other openings that provide a risk of herniated nerves.

The embodiments described herein are exemplary. Modifications, rearrangements, substitute processes, alternative elements, etc. may be made to these embodiments and still be encompassed within the teachings set forth herein.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements and/or states are in any way required for one or more embodiments. The terms “comprising,” “including,” “having,” “involving,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y or Z,” unless specifically stated otherwise, is otherwise understood with the context as used in general to present that an item, term, etc., may be either X, Y or Z, or any combination thereof (e.g., X, Y and/or Z). Thus, such disjunctive language is not generally intended to, and should not, imply that certain embodiments require at least one of X, at least one of Y or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

While the above detailed description has shown, described, and pointed out novel features as applied to illustrative embodiments, it will be understood that various omissions, substitutions, and changes in the form and details of the devices illustrated can be made without departing from the spirit of the disclosure. As will be recognized, certain embodiments described herein can be embodied within a form that does not provide all of the features and benefits set forth herein, as some features can be used or practiced separately from others. All changes which come within the meaning and range of equivalency of the claims are to be embraced within their scope.

What is claimed is:

1. A clip for dural closure, comprising:
a base;
a first leg extending distally from the base and comprising a first tip, the first tip comprising a first plurality of digits;
a second leg extending distally from the base and comprising a second tip, the second tip comprising a second plurality of digits; and
a sealant positioned on an underside of the base and configured to provide a fluid seal along at least a portion of a dural opening;
wherein the clip is transformable between an open configuration and a closed configuration, wherein the first plurality of digits of the first tip of the first leg are configured to interdigitate with the second plurality of digits of the second tip of the second leg in the closed configuration.

2. The clip of claim 1, wherein each digit of the first plurality of digits comprises a protrusion configured to be received within a depression of the second plurality of digits.

3. The clip of claim 2, wherein the protrusion is convex, and the depression is concave.

4. The clip of claim 1, wherein the first tip and the second tip are configured to clip tissue on opposing sides of the dural opening in the closed configuration to seal at least a portion of the dural opening without piercing or perforating the tissue.

5. The clip of claim 4, wherein each digit of the first plurality of digits comprises an atraumatic distal end and each digit of the second plurality of digits comprises an atraumatic distal end.

6. The clip of claim 4, wherein each digit of the first plurality of digits comprises a rounded distal end and each digit of the second plurality of digits comprises a rounded distal end.

7. The clip of claim 1, wherein the first plurality of digits comprises n digits and the second plurality of digits comprises n+1 digits.

8. The clip of claim 1, wherein the sealant comprises a collagen matrix.

9. The clip of claim 1, wherein the sealant comprises a plug coupled to the underside of the base.

10. The clip of claim 1, wherein the sealant extends along an entirety of the underside of the base between the first leg and the second leg.

11. The clip of claim 1, wherein the sealant comprises an adhesive material.

12. The clip of claim 1, wherein the clip is bioabsorbable.

13. The clip of claim 1, wherein the clip comprises a shape memory material.

14. The clip of claim 1, wherein the clip is biased to the closed configuration.

15. The clip of claim 1, wherein the clip comprises a first catch and a second catch, each of the first catch and the second catch configured to be grasped by a clip delivery device to transition the clip from the closed configuration to the open configuration.

16. The clip of claim 15, wherein the first catch is positioned on the first leg and the second catch is positioned on the second leg.

17. The clip of claim 1, wherein a width of a proximal end of the clip is no more than 1.5 times a width of a distal end of the clip.

18. The clip of claim 1, wherein each digit of the first plurality of digits is angled distally.

19. The clip of claim 1, wherein each digit of the first plurality of digits extends distally from a base of the digit to an apex and extends proximally from the apex to a distal end of the digit.
